(19) United States
(12) Patent Application Publication
(10) Pub. No.: US 2024/0358731 A1
(43) Pub. Date: Oct. 31, 2024

(54) SUMO PATHWAY AS AN OXYGEN SENSOR RELEVANT TO THE PATHOPHYSIOLOGY OF PULMONARY ARTERY HYPERTENSION AND CARDIAC ARRHYTHMIA

(71) Applicant: Northeastern University, Boston, MA (US)

(72) Inventor: Leigh D. Plant, Boston, MA (US)

(21) Appl. No.: 18/640,725

(22) Filed: Apr. 19, 2024

Related U.S. Application Data
(60) Provisional application No. 63/460,365, filed on Apr. 19, 2023.

(51) Int. Cl. A61K 31/7034; A61P 9/12

(52) U.S. Cl. CPC A61K 31/7034; A61P 9/12

(57) ABSTRACT

Disclosed are methods of treating or preventing a cardiovascular disease. The method may comprise administering to a subject in need thereof an effective amount of a compound. The compound may be a SUMOylation inhibitor, an analogue of Phosphatidylinositol 4,5-bisphosphate (PIP2); PIP2; or a compound that increases endogenous PIP2.

### RELATED APPLICATIONS

This application claims the benefit of priority to U.S. Provisional Patent Application Ser. No. 63/460,365, filed Apr. 19, 2023.

### GOVERNMENT SUPPORT

This invention was made with government support under Grant No. R01HL144615 awarded by the National Institutes of Health. The government has certain rights in the invention.

### REFERENCE TO A SEQUENCE LISTING XML

This application contains a Sequence Listing which has been submitted electronically in XML format. The Sequence Listing XML is incorporated herein by reference. Said XML file, created on Jul. 12, 2024, is named NEX-14701_SL.xml and is 11,211 bytes in size.

### BACKGROUND

Cardiovascular diseases (CVDs) are a group of disorders of the heart and blood vessels and include pulmonary artery hypertension, arrhythmia, coronary heart disease, cerebrovascular disease, rheumatic heart disease, and other conditions. Cardiovascular diseases are the number one cause of death globally, taking an estimated 17.9 million lives each year. Four out of five CVD deaths are due to heart attacks and strokes, and one third of these deaths occur prematurely in people under 70 years of age. Individuals at risk of CVD may demonstrate raised blood pressure, glucose, and lipids as well as overweight and obesity. Given that cardiovascular diseases are a leading cause of death throughout the world, there remains a need for therapies that can be used to treat cardiovascular diseases.

### SUMMARY

Disclosed are methods of treating or preventing a cardiovascular disease. The method may comprise administering to a subject in need thereof an effective amount of a compound. The compound may be a SUMOylation inhibitor, an analogue of Phosphatidylinositol 4,5-bisphosphate (PIP2); PIP2; or a compound that increases endogenous PIP2.

In some embodiments, the compound is a SUMOylation inhibitor; and the compound is topotecan, nocardione A, 33-DINOR-dunnione, 33-DINOR-dehydrodunnione, and β-lapachone, macrophilone A, compound 61, Triptolide, or N106. In some embodiments, the compound is a SUMOylation inhibitor; and the compound is an inhibitor of SAE1. In some embodiments, the inhibitor of SAE1 is ginkgolic acid, anacardic acid, Kerriamycin B, Davidiin, tannic acid, SUMO-AMSN, SUMO-AVSN, phenyl urea compounds, compound 10, pyrazole urea, thiazole urea, CID9549553, COH000, compound 15, ML-792, pevoncdistat, or TAK-981. In some embodiments, the compound is a SUMOylation inhibitor; and the compound is an inhibitor of SAE2. In some embodiments, the inhibitor of SAE2 is spectomycin B1, chactochromin A, viomellein, flavone 2-D08, compound 22, GSK145A, compound 24, compound 25, compound 26, or compound 27. In some embodiments, the compound is a SUMOylation inhibitor; and the compound is an inhibitor of SENP1. In some embodiments, the inhibitor of SENP1 is momordin Ic, streptonigrin, NSC76919, NSC45384, vialinin A, atromentin, JCP-666, VEA-260, VAE-499, VAE-500, VAE-561, compound 39, GN6767, GN6958, compound 42, compound 43, SPI-01, SPI-02, compound 46, compound 47, SI2, compound 49, compound 50, or compound 51. In some embodiments, the compound is a SUMOylation inhibitor; and the compound is an activator of SENP1.

In some embodiments, the compound increases endogenous PIP2; and the compound is an inhibitor of a Gαq-coupled AT1 receptor. In some embodiments, the inhibitor of a Gαq-coupled AT1 receptor is losartan, Exp 3174, telmisartan, irbesartan, candesartan, valsartan, cprosartan, azilsartan, saprisartan or olmesartan. In some embodiments, the compound increases endogenous PIP2; and the compound is an inhibitor of phospholipase C (PLCβ). In some embodiments, the inhibitor of PLCβ is U73122, phenylmethylsulfonyl fluoride, manoalide, D609, ET-18-OCH3, compound 48/80 trihydrochloride, spermine tetrahydrochloride, neomycin sulfate, NCDC, or thielavin B. In some embodiments, the compound is an analogue of PIP2; and the PIP2 analogue is diC8-PIP2. In some embodiments, the compound increases endogenous PIP2; and the compound inhibits PIP2 hydrolysis or inhibits PIP2 dephosphorylation.

In some embodiments, the cardiovascular disease is a hypertension, arrhythmia, coronary heart disease, cerebrovascular disease, rheumatic heart disease, ventricular hypertrophy, heart failure, vasculitis, atherosclerosis, myocardial infarction, angina pectoris, renal failure, transient ischemic attacks, peripheral vascular disease, aneurysm formation, hypercholesterolemia, hyperlipidemia, hyperlipoproteinemia, deep vein thrombosis, ventricular arrythmia, supraventricular tachycardia, or platelet aggregation. In some embodiments, the hypertension is pulmonary artery hypertension, systemic hypertension, pulmonary hypertension, sporadic pulmonary arterial hypertension, familial pulmonary arterial hypertension, idiopathic pulmonary arterial hypertension, or acquired pulmonary arterial hypertension. In some embodiments, the patient has a disease that reduces blood oxygen levels. In some embodiments, the patient has an infection, inflammation, sepsis, cystic fibrosis, COPD, sleep apnea, or cancer.

### BRIEF DESCRIPTION OF THE DRAWINGS

FIGS. 1A-1C show that acute hypoxia inhibits I_{K1} in cardiomyocytes via SUMOylation. I_{K1} in rat ventricular cardiomyocytes studied by whole-cell patch-clamp. Hypoxia was a drop in O₂ from ambient levels to 2%, measured at the cell. The time-course of hypoxic-inhibition was studied by recording the magnitude of I_{K1} every second. Cells were studied with the following pipette solutions: control, SUMO1 (1 μM) or SENP1 (2 μM). I_{K1} was blocked by the addition of 3 mM Ba²⁺. FIG. 1A shows that exposure to acute hypoxia (2% O₂) inhibits ˜40% of I_{K1}. Left, representative current sweeps; right, a representative time course showing the kinetics of hypoxic inhibition in cardiomyocytes studied with control pipette solution. The inhibition is precluded by including SENP1 (2 μM, magenta) in the recording pipette. The reverse ramp recording protocol is inset. FIG. 1B shows that including SUMO1 (1 μM) in the recording pipette inhibits I_{K1} and precludes the effects of hypoxia. SENP1 augments I_{K1} and protects the current from hypoxia. Data are from 8-10 RVCMs per group, **** P<0.0001, paired, two-tailed Student's 1-test. FIG. 1C shows PLA showing that native Kir2.1 colocalizes with SUMO1 in cardiomyocytes and that interactions are increased by acute hypoxia. Kir2.2 and Kir2.3 do not associate with SUMO1. Representative data are shown with DAPI labeled nuclei and PLA interactions. Summary data are obtained from multiple experiments each with multiple fields of view containing 20-30 nuclei. The scale bar is 10 μm.

FIG. 2 shows that the SUMO pathway showing steps required for covalent modification of a target protein. SUMOylation is reversed by SENPs.

FIGS. 3A-3D show that hypoxia diminishes Kir2.4 activity in a TAK981 sensitive manner. FIG. 3A shows representative trace of Kir2.4 channel current following acute hypoxia. FIG. 3B shows summary data showing the current densities of Kir2.4 channels in HEK cells. FIG. 3C shows that representative trace of Kir2.4 channel current is unresponsive to hypoxia after pre-treatment with TAK981 FIG. 3D shows summary data showing that the effect of hypoxia is abolished in cells that were pre-treated with TAK981.

FIGS. 4A-4D show that PIP2 opposed hypoxic-inhibition of I_{K1}. To knockdown Kir2.1 expression, RVCMs were transduced with lentiviral particles carrying cGFP and shRNA targeting KCNJ2-mRNA for the channel. Kir2.1 knockdown cells (Kir2.1kd-CMs) were identified by expression of cGFP. Cells were studied with control pipette solution, or with pipette solutions containing purified SUMO1 (1 μM) or SENP1 (2 μM). Where indicated, the control pipette solution contained diC8 PIP₂. Paired patch-clamp data were analyzed by Students t-test; ***, P<0.01. FIG. 4A Left shows example sweeps showing that I_{K1} is diminished in Kir2.1kd-CMs and the remaining current is insensitive to acute hypoxia. Kir2.1kd-CMs are identified by expression of GFP (inset, scale bar=10 μm). Right, summary data from 8-10 cells per group show that regulation of I_{K1} by hypoxia, SUMO1 and SENP1 is lost in Kir2.1kd-CMs. FIG. 4B shows that the magnitude and the regulation of I_{K1} by hypoxia, SUMO1 and SENP1 is unaltered when cells are treated with a control, scrambled shRNA; 7-8 cells per group. FIG. 4C, Left, shows example traces to show that including diC8-PIP2 in the pipette solution reduces the hypoxic-inhibition of I_{K1} in RVCMs in a concentration-dependent manner. The arrow indicates the change in current-magnitude between exposure to ambient O₂ and 2% O₂ in the same cell. Right, Concentration-response curve showing that including diC8-PIP2 in the pipette opposes hypoxic-inhibition of I_{K1} in control RVCMs and RVCMs expressing the scrambled shRNA. The hypoxic-response is diminished in Kir2.1kd-CMs. Data are mean±s.d. for 7-8 cells per condition. FIG. 4D shows that DiC8-PIP2 decreases hypoxic-inhibition of Kir2.1 channels expressed in HEK293T cells (control) in a concentration-dependent manner using the experimental paradigm described in FIG. 4C, above. Current inhibition is diminished when SENP1 is included in the recording pipette and is not observed when SUMO-insensitive Kir2.1-K49Q channels are studied. Data are mean±s.d. for 6 cells per condition.

FIGS. 5A-5D shows that co-expressed SUMO decreases Kir2.4 currents. FIG. 5A shows that human Kir2.1 and 2.4 channels contain a high probability SUMO-motif. FIG. 5A discloses SEQ ID NOS 6-9, respectively, in order of appearance. FIGS. 5B-5C shows that co-injection of SUMO1 decreases Kir2.1 and 2.4 currents in a dose-dependent manner, assessed by TEVC at −80 mV in 96 mM external K+. SENP1 increases the current. FIG. 5D shows that Kir2.3 has a low probability SUMO-motif at the same site and is insensitive to SUMO1 and SENP1. SUMO sensitivity is transferred to Kir2.3 when the site in Kir2.1 is recreated by mutagenesis (Kir2.3-N25D).

FIGS. 6A-6C show donor-decay FRET experiments. FIG. 6A shows representative traces of fluorescence decay of mTFP (donor) tagged Kir2.4 in the presence of free YFP (acceptor) compared to YFP-tagged SUMO1. FIGS. 6B, 6C show summary data for time constant of fluorescence decay (tau) showing FIG. 6B, mTFP-Kir2.4 FRETs with YFP-SUMO1 FIG. 6C, but not with mTFP-Kir2.4-K54Q.

FIG. 7 shows whole-cell patch clamp recording with inclusion of 1 nM SUMO1 or SENP1 peptide in the pipette shows that human Kir2.1 and Kir2.4, but not Kir2.3 pass SUMO-regulated currents. Currents were measured at −80 mV in 140 mM external K+.

FIGS. 8A-8B show activation of the optogenetic PIP₂ 5-ptase, 5-Ptase_{OCRL}, with 460 nm light reduced Kir2 currents. FIG. 8A shows that example Kir2.4 sweeps before and after activation of 5-Ptase_{OCRL}. The current is sensitive to Ba²⁺. Example time-course showing that the decrease in Kir2.4 current requires activation of 5-Ptase_{OCRL} (upper). FIG. 8B shows summary data showing the %-inhibition of Kir2.1, 2.3 and 2.4 with 5-min activation of 5-Ptase_{OCRL}.

FIG. 9 shows that whole-cell 2.4 currents are no longer decreased by inclusion of 1 nM SUMO1 in the recording pipette when cells are studied following 5 min of continuous activation of 5-Ptase_{OCRL}, depleting PIP2. Similarly, currents were no longer enhanced by SENP1.

FIGS. 10A-10B show whole cell patch clamp recording of human pulmonary artery smooth muscle cells. FIG. 10A LEFT shows example records to show, in the same hPASMC that the inward current is decreased by a transition from ambient O₂ to acute hypoxia. RIGHT: Pretreating the hPASMCs with 100 nM TAK981 does not alter the magnitude or the phenotype of the initial current in ambient O₂ (blue), but precludes the change in the current caused by acute hypoxia. FIG. 10B LEFT shows summary data to show that in 5 separate studies, the magnitude of the hPASMC inward current decreased upon exposure to acute hypoxia. The decrease is statistically significant, *, P<0.05 by Students t-test. RIGHT: Summary data to show that pretreatment with 100 nM TAK981 precludes the change in the current caused by acute hypoxia.

### DETAILED DESCRIPTION

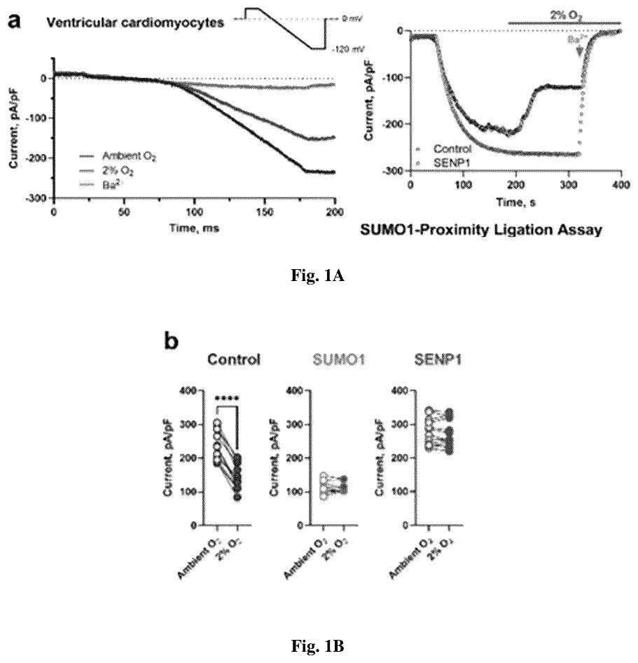

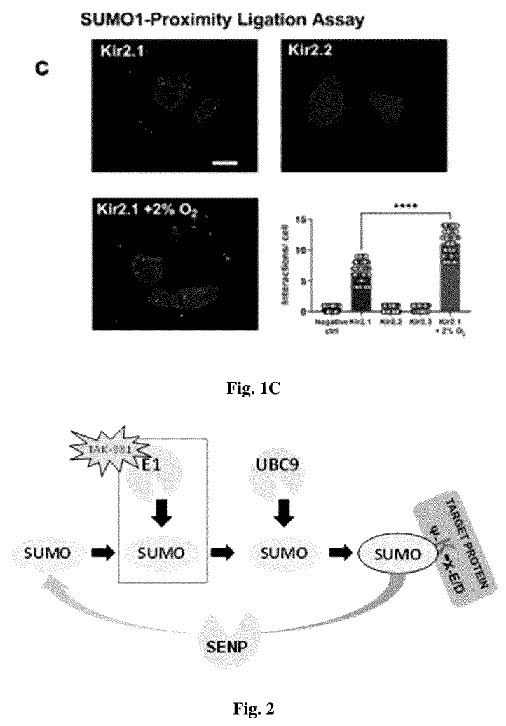

Pulmonary vessels constrict in response to acute hypoxia, changing the blood flow in favor of areas of the lung that can better support oxygenation. This adaptive response occurs because pulmonary artery smooth muscle cells (PASMCs) are intrinsically sensitive to hypoxia and contract when O₂ levels fall. Maladaptive pulmonary physiology, cardiovascular diseases, drugs such as halogenated anesthetics, or infections such as COVID can evoke sustained hypoxic pulmonary vasoconstriction (HPV) and the downstream proliferation of PASMCs. In primary arterial hypertension (PAH), this remodeling process narrows the arteries, increasing vascular resistance. Despite symptomatic management, severe PAH can progress to right side heart failure with a median survival rate of under three years. The prevalence and prognosis of PAH is worse in the elderly, newborns and women and is further exacerbated by urban-rural disparities, misdiagnosis, misclassification of the form of hypertension, and comorbidities. Although much is understood about the physiology of HPV, the identity of the O₂ sensor in PASMCs remains enigmatic. Relevant K⁺ channels are inhibited by acute hypoxia, causing PASMCs to contract. However, it is unclear if the channels themselves sense hypoxia or if the channels are regulated by cellular signaling processes. Many of the K⁺ channels proposed to be expressed in PASMCs are directly regulated by the SUMO (small ubiquitin-related modifier) pathway. SUMOylation is an enzyme-mediated post-translational modification (PTM) that results in the covalent attachment of a ˜ 100 amino acid SUMO peptide (SUMOs 1, 2 or 3) to key lysine residues in target proteins. We have shown that SUMOylation is a rapid, reversible regulator of specific ion channels that is enhanced by acute hypoxia, identifying the pathway as a candidate O₂-sensor. The inward rectifying K⁺ channel, Kir2.1 is regulated by the interplay of SUMOylation and the membrane phospholipid phosphatidylinositol 4,5-bisphosphate (PIP2). Thus, acute hypoxia diminishes the Kir2.1-mediated I_{K1} current in cardiomyocytes because the channels are rapidly SUMOylated (FIG. 1). SUMO-binding to Kir2.1 reduces the potency and efficacy of PIP2 to gate the channel open. We investigated the effect of SUMOylation on K⁺ channels in human PASMCs, with a focus on Kir2s. Prior studies show that PASMCs express Kir2.1 but have robust expression of Kir2.4. Encoded by the kcnj14 gene, Kir2.4 is an understudied, PIP2-sensitive channel that we show is SUMO-regulated like Kir2.1. Our studies take into consideration that other channels are likely also subject to this regulatory mechanism. We show that hypoxia-induced SUMO-inhibition of PASMC K⁺ channels is a PIP2 dependent mechanism to mediate cellular contraction. This also shows the utility of small-molecule inhibitors of the SUMO pathway to alleviate prolonged HPV. In support of this, our data show that hypoxia-induced SUMOylation of K⁺ channels is ameliorated by TAK981, a drug currently in clinical trials for solid tumors.

PASMCs contract in response to acute local hypoxia, constricting the pulmonary vasculature to increase blood flow to areas of the lung that allow better oxygenation. Chronic hypoxia, secondary to cardiovascular or pulmonary diseases, infection or tissue damage can sustain hypoxic pulmonary vasoconstriction (HPV), remodel the physiology of PASMCs and precipitate progressive PAH. Although significant progress has been made to unravel the physiological basis for HPV, the O₂-sensor in PASMCs has been elusive, hampering drug development efforts. Elegant studies have proposed that multiple K⁺ channels, including K_{V}1.5, K_{V}2.1 and K2P3 act as O₂-sensors in PASMCs, usually from animal models. Human PASMCs also express Kir2 channels, with RT-PCR showing Kir2.4 as the most dominant transcript. Hypoxic inhibition of K⁺ currents depolarize PASMCs, activating Ca²⁺ channels and promoting contraction. Although several K⁺ channels are also inhibited by acute hypoxia in expression systems, their role as direct O₂-sensors is less well established and is proposed to be secondary to an increase in free-radical production. We highlight that this same set of channels are (i) inhibited by SUMOylation, and (ii) regulated by interaction with the phospholipid, PIP2.

SUMOylation of ion channels: Classically considered as a nuclear signal, the SUMO pathway is associated with cardiovascular development, metabolism, and stress responses. A growing number of ion channels are recognized to be regulated by SUMOylation, including K2Ps, K_{V}1.5, K_{V}2.1 and K_{V}7s, Kir2.1, Na_{V}1.2 and Na_{V}1.5. However, the mechanistic basis for changes in SUMOylation, or the action of SUMO proteins on the dynamics and kinetics of ion channels remain poorly understood. We showed that hypoxia decreases Kir2.1 currents in cardiomyocytes, due to a rapid increase in channel SUMOylation. This shows that SUMO regulates Kir2 channels and shows activation of the pathway by hypoxia. Thus, suppression of Kir2.1 activity is mimicked by excess exogenous SUMO1 and is prevented when the deSUMOylating enzyme, SENP1 (SUMO specific proteases) is introduced to the cells via the patch-pipette. Although SUMOylation is covalent, it is reversible via the action of SENP enzymes. Indeed, exogenous SENP1 renders I_{K1} in cardiomyocytes insensitive to acute hypoxia (FIG. 1). SUMOs attach to intracellular lysines in target proteins, at the second position of the consensus motif ψ-K-X-E/D. Using sequence analysis, we found that the ‘SUMOylation-motif’ in Kir2.1 is shared with Kir2.4 but not Kir2.2 and Kir2.3, making it likely that Kir2.4 channels (not expressed in cardiomyocytes) are also suppressed by hypoxia induced SUMOylation. The possibility that pulmonary vessels sense hypoxia via a similar mechanism is intriguing and motivated our studies into the regulation of Kir2.4. TAK981 (Subasumstat) is a first-in-class nM-inhibitor of the SUMO-activator enzyme SAE1 (or E1) (FIG. 2). This highly selective ligand is in clinical trials to treat solid tumors. When coupled to the E1 SUMO-activating enzyme, TAK981 forms an irreversible adduct with each of the three functional mammalian SUMO paralogues (SUMO1, SUMO2, & SUMO3) which occupies the E1 active site, blocking the transfer of SUMO to the SUMO conjugating enzyme, UBC9 and eventual ligation of SUMO to substrate proteins (FIG. 2). Pharmacological suppression of SUMOylation with TAK981 promotes adaptive antitumor immune responses by activating IFN1 signaling in T cells and dendritic cells. TAK981 has solidified its candidacy as an antineoplastic agent by significantly augmenting the anticancer activity of the therapeutic antibodies. TAK981, is currently in phase 2-clinical trial for its anti-cancer activity but is repurposed here with the proof of principle goal of enhancing the landscape of PAH therapies with a targeted approach toward a pathology-based solution. Our study explores repurposing TAK981 as an in vitro proof-of-concept molecule, transforming its utility as anti-cancer agent and diversify its application to a sub-acute process like hypoxia with significant preventative clinical upside.

This study provides a new understanding of important regulatory pathways: SUMO-regulation of Kir2.4 channels in PASMCs, its underlying PIP2-regulation, and the potential reversal of this dysfunction by TAK981. We present data to support these assertions, as well as evidence of a hypoxia sensitive mechanism that perpetuates Kir2.4 SUMOylation.

We have employed patch-clamp electrophysiology to investigate ion channel currents within cells. When subjected to acute hypoxia, we observe alterations in the activity of particular channels or channel groups. We then employ techniques to isolate the influence of the SUMO pathway, demonstrating its regulatory role. Additionally, we can relate these changes to the overall physiology of muscle cells, including their responsiveness to hypoxic conditions, such as their contractile capacity.

This method will, for first time, provide that the SUMO pathway senses acute changes in oxygen. Once activated, SUMOylation initiates the cellular response via several effector ion channel types, producing a synergistic effect (signal amplification). The SUMO pathway can be blocked in several ways, preventing the oxygen sensing effect and the downstream physiological/pathophysiological changes. The same oxygen sensing mechanisms are active in other relevant cells, such as cardiac myocytes and specific types of neurons.

This method will, for first time, provide identification of the acute oxygen sensing mechanism in pulmonary cells that then allows the cells to contract. Further, this method will, for first time, provide identification of the oxygen sensing mechanism that can be targeted to prevent arrhythmia when cardiac muscle becomes hypoxic.

This method will, for first time, provide targeting drugs to the SUMO pathway as anti-arrhythmics, targeting drugs to the SUMO pathway to prevent acute changes in pulmonary artery hypertension, and targeting drugs to the SUMO pathway to prevent changes in relevant cells during disease states that reduce oxygen levels, such as infection, inflammation, sepsis, and cancer.

This method will, for first time, provide delivery of a SUMO inhibitor molecule to a specific tissue, for example to develop a known experimental SUMO inhibitor as an aerosol (inhaler) that could be used to deliver the drug to blood vessels in the lung for pulmonary artery hypertension.

This method will, for first time, provide ad hoc use for in-patient critical care with sepsis or a high burden of inflammation/infection, treat hypoxic response due to infections in airways of patients with cystic fibrosis, and patients with low level chronic hypoxia, such as COPD/sleep apnea.

Disclosed are methods of treating or preventing a cardiovascular disease. The method may comprise administering to a subject in need thereof an effective amount of a compound. The compound may be a SUMOylation inhibitor, an analogue of Phosphatidylinositol 4,5-bisphosphate (PIP2); PIP2; or a compound that increases endogenous PIP2.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Unless specifically stated or obvious from context, as used herein, the term “or” is understood to be inclusive. Unless specifically stated or obvious from context, as used herein, the terms “a”, “an”, and “the” are understood to be singular or plural.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

For the purposes of this invention the term “therapeutically effective amount” of a therapeutic refers to the amount of the therapeutic which, when administered to a subject, elicits adequate therapeutic response in the subject to provide beneficial therapeutic outcome in the subject. A therapeutically effective amount may vary depending upon the intended application; the subject being treated, e.g., the weight and age of the subject; disease condition being treated, e.g., the severity of the disease condition; and the manner of administration. Based on these factors, a skilled artisan can determine a therapeutically effective amount of a therapeutic in a given situation.

As used herein, the term “administering” means providing a therapeutic agent or composition to a subject, and includes, but is not limited to, administering by a medical professional and self-administering. The means of providing a therapeutic agent are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated,” for example, if one or more symptoms associated with a particular disease, disorder, or condition are mitigated or eliminated.

In certain embodiments, a therapeutic agent may be used alone or conjointly administered with another therapeutic agent. As used herein, the phrase “conjoint administration” refers to any form of administration of two or more different therapeutic agents such that the second agent is administered while the previously administered therapeutic agent is still effective in the body (e.g., the two agents are simultaneously effective in the subject, which may include synergistic effects of the two agents). For example, the different therapeutic agents can be administered either in the same formulation or in separate formulations, either concomitantly or sequentially. In certain embodiments, the different therapeutic agents can be administered within about one hour, about 12 hours, about 24 hours, about 36 hours, about 48 hours, about 72 hours, or about a week of one another. Thus, a subject who receives such treatment can benefit from a combined effect of different therapeutic agents.

In certain embodiments, conjoint administration of the combinations of compounds of the invention with one or more additional therapeutic agent(s) (e.g., one or more additional chemotherapeutic agent(s)) provides improved efficacy relative to each individual administration of the combinations of compounds of the invention or the one or more additional therapeutic agent(s). In certain such embodiments, the conjoint administration provides an additive effect, wherein an additive effect refers to the sum of each of the effects of individual administration of the combinations of compounds of the invention and the one or more additional therapeutic agent(s).

The term “a small molecule” is a compound having a molecular weight of less than 2000 Daltons, preferably less than 1000 Daltons. Typically, a small molecule therapeutic is an organic compound that may help regulate a biological process.

“Subject” refers to an animal, such as a mammal, for example a human. The methods described herein can be useful in both humans and non-human animals. In some embodiments, the subject is a mammal (such as an animal model of disease), and in some embodiments, the subject is human.

The term “activator” refers to a compound having the ability to inhibit a biological function of a target biomolecule, for example, an mRNA or a protein, whether by increasing the activity or expression of the target biomolecule. Accordingly, the term “activator” is defined in the context of the biological role of the target biomolecule.

### Methods

The blood gets oxygenated in the lungs. Yet, if oxygen levels drop too low in a lung region, hindering this process, specialized muscle cells in the pulmonary blood vessels contract. This contraction redirects blood to healthier lung areas. Despite its significance, its mechanism is not known. Understanding it could prevent illnesses like primary artery hypertension (PAH).

PAH occurs when lung blood vessels remain constricted due to various factors like disease, medication side effects, or infection, leading to muscle cell proliferation, vessel narrowing, and increased blood pressure. It is prevalent among the elderly, women, and newborns, posing severe health risks.

How do these muscle cells sense low oxygen levels? Some research indicates that low oxygen alters ion flow across cell membranes, implicating ion channel proteins as potential oxygen sensors. However, this theory faces challenges. Our findings show that the SUMO pathway detects oxygen levels, becoming activated during oxygen depletion. SUMO then modulates specific ion channels, prompting muscle cell contraction. Our study opens avenues for new scientific insights and could aid in developing drugs to combat PAH. Notably, similar mechanisms operate in cardiomyocytes, linking pathway activation to cardiac arrhythmias.

Disclosed are methods of treating or preventing a cardiovascular disease. The method may comprise administering to a subject in need thereof an effective amount of a compound. The compound may be a SUMOylation inhibitor, an analogue of Phosphatidylinositol 4,5-bisphosphate (PIP2); PIP2; or a compound that increases endogenous PIP2.

### SUMOylation Inhibitors

SUMOylation is a post-translational modification that involves the covalent attachment of the small ubiquitin-like modifier (SUMO) polypeptide to a lysine residue of a target protein. The enzymatic pathway of SUMOylation is very similar to ubiquitinylation and involves an activating enzyme, a conjugating enzyme, ligases and deconjugating enzymes. SUMOylation modulates the function of a number of proteins associated with various pathways.

SUMOylation is a dynamic and reversible process that is controlled by an enzymatic cascade very similar to ubiquitinylation. It requires an ATP-dependent heterodimeric SUMO-activating enzyme dubbed E1, a SUMO conjugating enzyme, E2, and E3 ligases that assist in the recognition of substrates and increase the efficiency of isopeptide formation. Finally, SUMO/sentrin specific peptidases (SENPs) hydrolyze SUMO from the substrate, reversing SUMOylation.

There is one human SUMO E2 enzyme, Ubc9, six SENPs and ten SUMO E3 ligases. Three isoforms of SUMO (SUMO1-3) are expressed in humans, and there are key differences among them. SUMO2 and SUMO3 share 97% sequence homology and are often denoted as SUMO2/3 as many detection methods cannot distinguish between them.

The first step in the SUMOylation pathway is maturation of the SUMO protein. Two isoforms of SENPs, SENP1 and SENP2, are responsible for cleaving the tetrapeptide C-terminal cap to reveal a di-glycine motif. The di-glycine motif eventually forms the isopeptide bond with the ε-amino group of a lysine residue on the protein substrate. After SUMO maturation has occurred, the E1 enzyme will activate the protein for conjugation. The SUMO activating enzyme (SAE) is a heterodimeric protein complex that consists of Aos1/SAE1 and Uba2/SAE2. Activation of mature SUMO protein is ATP-dependent and involves two discrete reactions. The first step is adenylation of the C-terminus of SUMO, which consumes ATP and releases pyrophosphate as a byproduct. The catalytic cysteine in the E1 enzyme attacks the acylated-adenylate to form a thioester intermediate. After the E1-SUMO complex is formed, SUMO is then transferred to a cysteine residue on the SUMO-conjugating enzyme, SUMO E2, through a transthioesterification reaction. SUMO E2 catalyzes formation of the isopeptide bond between the side chain amine of lysine and the C-terminal glycine residue of SUMO, often with the assistance of an E3 ligase, which can increase the rate of SUMOylation. E3 ligases are also involved in colocalization of the E2-SUMO complex and the target protein. However, it has been demonstrated that SUMOylation can occur without the use of an E3 ligase, which is the case for RanGAP1, one of the first SUMO substrates identified.

### SUMO-E1 Inhibitors:

SUMO activating enzyme (SAE) is responsible for the first step in the SUMOylation enzymatic cascade. Several natural products have been identified as SUMO-E1 inhibitors. The first identified inhibitors of SUMOylation was ginkgolic acid (1), and its analog, anacardic acid (2). These molecules inhibit SAE by blocking formation of the E1-SUMO complex. Kerriamycin (3) B blocks formation of the E1-SUMO complex. Kerriamycin B was first isolated from Streptomyces violaceolatus. Davidiin (4), isolated from the plant Davidia involucrate, is one of the most potent natural product SUMOylation inhibitors identified. A structurally similar compound, tannic acid, was identified through a gene expression assay as a nontoxic SUMOylation inhibitor. Like davidiin and ginkgolic acid, tannic acid (5) blocks the formation of the E1-SUMO complex.

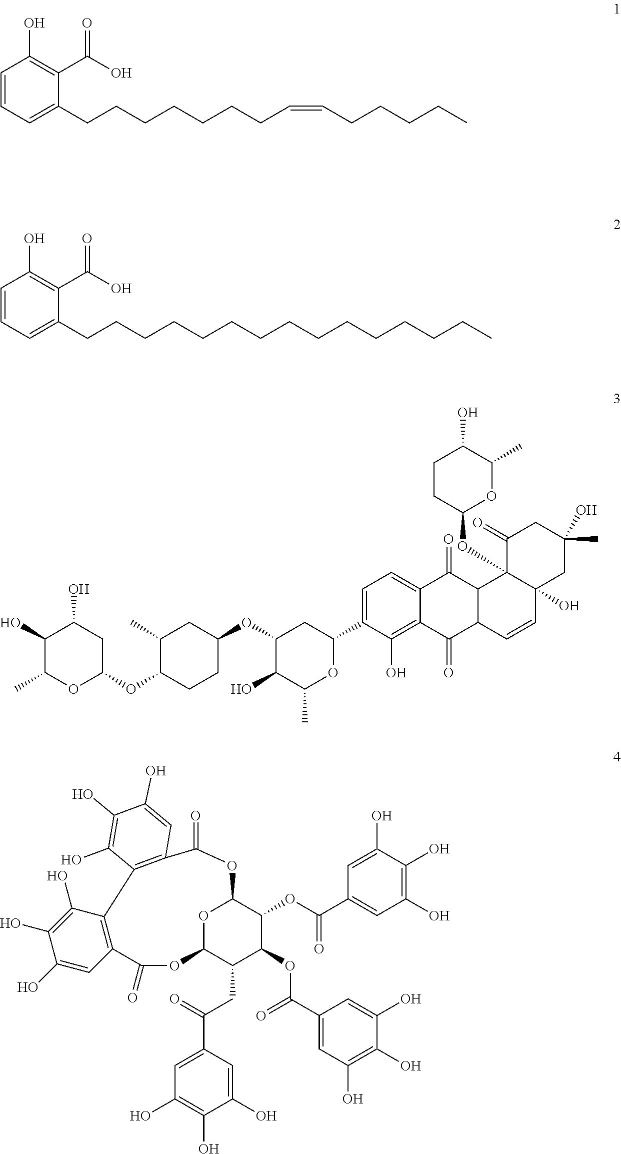

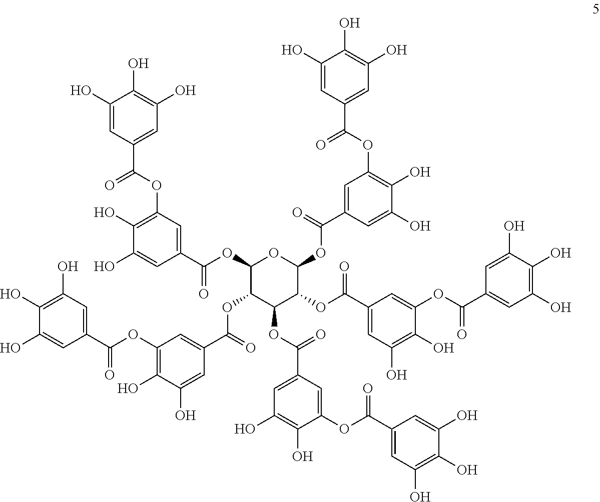 [SMILES: O=C(Oc1cc(C(=O)O[C@H]2O[C@@H](OC(=O)c3cc(O)c(O)c(OC(=O)c4cc(O)c(O)c(O)c4)c3)[C@H](OC(=O)c3cc(O)c(O)c(OC(=O)c4cc(O)c(O)c(O)c4)c3)[C@@H](OC(=O)c3cc(O)c(O)c(OC(=O)c4cc(O)c(O)c(O)c4)c3)[C@@H]2OC(=O)c2cc(O)c(O)c(OC(=O)c3cc(O)c(O)c(O)c3)c2)cc(O)c1O)c1cc(O)c(O)c(O)c1]

Employing native chemical ligation, CGG-AMSN and CGG-AVN were appended to truncated SUMO that was lacking the C-terminal diglycine residues to produce SUMO-AMSN (6) and SUMO-AVSN (7). Each ligand was designed to specifically inhibit the two half reactions catalyzed by SAE, as the vinyl sulfonamide can act as an electrophilic trap for the catalytic cysteine residues. These adducts were shown to selectively inhibit SAE over the analogous ubiquitin-activating enzyme and block formation of the E1-SUMO complex.

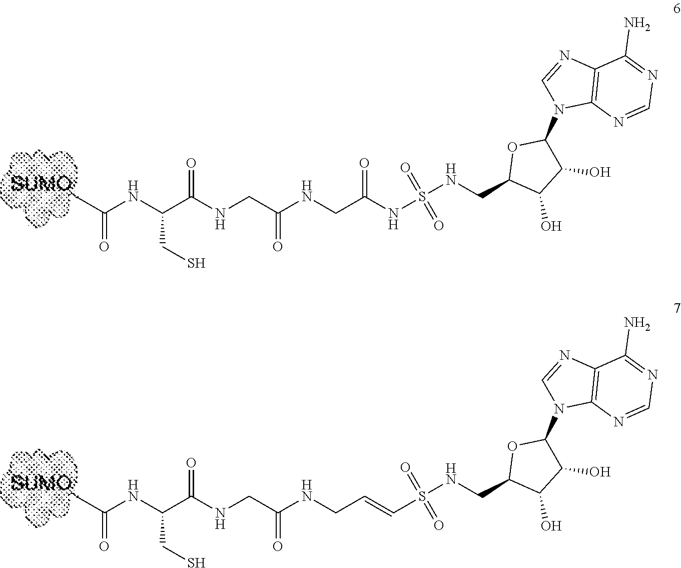 [SMILES: CC(=O)N[C@@H](CS)C(=O)NCC(=O)NCC(=O)NS(=O)(=O)NC[C@H]1O[C@@H](n2cnc3c(N)ncnc32)[C@H](O)[C@@H]1O ; CC(=O)N[C@@H](CS)C(=O)NCC(=O)NC/C=C/S(=O)(=O)NC[C@H]1O[C@@H](n2cnc3c(N)ncnc32)[C@H](O)[C@@H]1O]

Phenyl urea compounds (8 and 9) were identified as potential SUMOylation inhibitors. Compound 10 is a potent in vitro SUMOylation inhibitor. Compound 10 exhibits the same mode of activity as 8 and 9 and prevents formation of the E1-SUMO complex. Pyrazole urea (11) and thiazole urea (12) are potent molecules capable of inhibiting SUMOylation in vitro. CID9549553 (13) covalently targets an allosteric binding pocket of SUMO E1. Subsequent medicinal chemistry efforts led to a more potent SUMOylation inhibitor, COH000 (14). A class of tricyclic quinoxaline-based SAE inhibitors were identified wherein compound 15 was the most potent.

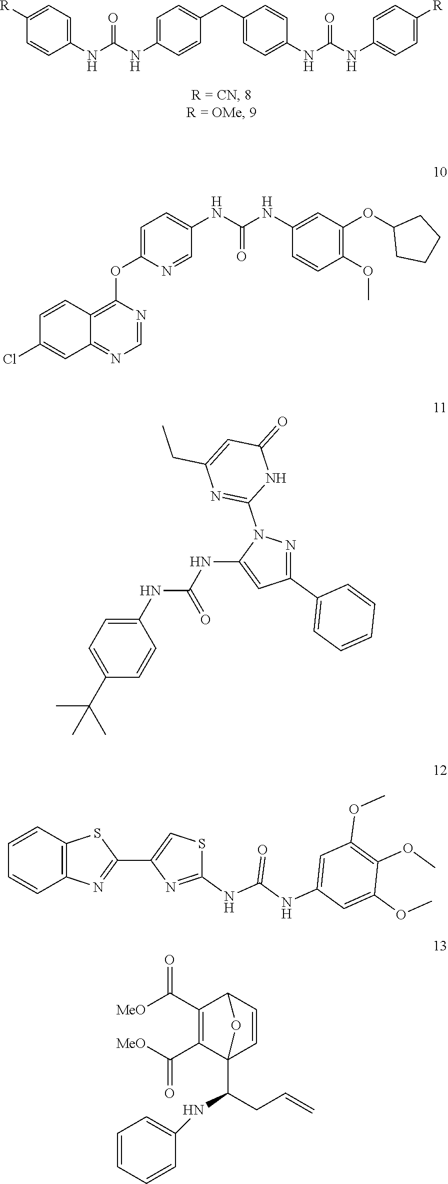

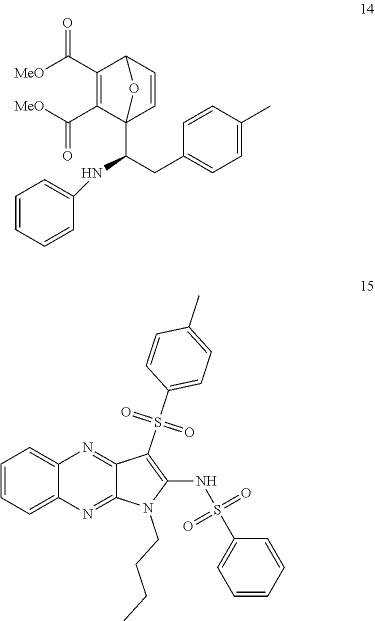 [SMILES: COC(=O)C1=C(C(=O)OC)C2([C@@H](Cc3ccc(C)cc3)Nc3ccccc3)C=CC1O2 ; CCCCn1c(NS(=O)(=O)c2ccccc2)c(S(=O)(=O)c2ccc(C)cc2)c2nc3ccccc3nc21]

Using pevonedistat as a starting point, researchers at Takeda Pharmaceuticals developed a selective SAE inhibitor, ML-792 (16). ML-792 manifests a similar mechanism of action as pevonedistat and forms a covalent adduct with SUMO that is catalyzed by SAE. An analog of 16, TAK-981 (17) is currently in Phase I clinical trials for the treatment of lymphomas and metastatic solid tumors.

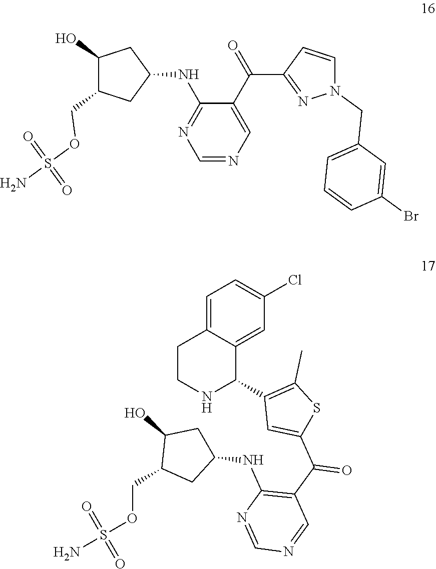 [SMILES: NS(=O)(=O)OC[C@H]1C[C@@H](Nc2ncncc2C(=O)c2ccn(Cc3cccc(Br)c3)n2)C[C@@H]1O ; Cc1sc(C(=O)c2cncnc2N[C@@H]2C[C@H](COS(N)(=O)=O)[C@@H](O)C2)cc1[C@@H]1NCCc2ccc(Cl)cc21]

### SUMO-E2 Inhibitors

After SUMO has been activated by the SAE, it is transferred to SUMO-E2, which catalyzes formation of the isopeptide bond between the C-terminal glycine residue of SUMO and the ≥-amino group of a lysine residue within the target protein. There is one E2 enzyme, Ubc9, that is responsible for conjugation within the SUMOylation pathway. As a result, there is an opportunity to inhibit the SUMOylation pathway by targeting one enzyme, as compared to the many enzymes required for ubiquitinylation.

Some of the first identified inhibitors of SUMO-E2 were the natural products spectomycin B1 (18) and structurally related compounds, chaetochromin A (19) and viomellein (20). Flavone 2-D08 (21) inhibits Ubc9 by preventing the transfer of SUMO from Ubc9 to the substrate. Using fluorescently labeled Ubc9 and UbcH5b, an analogous ubiquitin E2, 133 potential Ubc9 inhibitors were identified. Compound, 22 has modest selectivity for the SUMOylation pathway. Compound 22 was unable to block formation of the E1-SUMO complex, confirming that it manifests its activity via the inhibition of Ubc9.

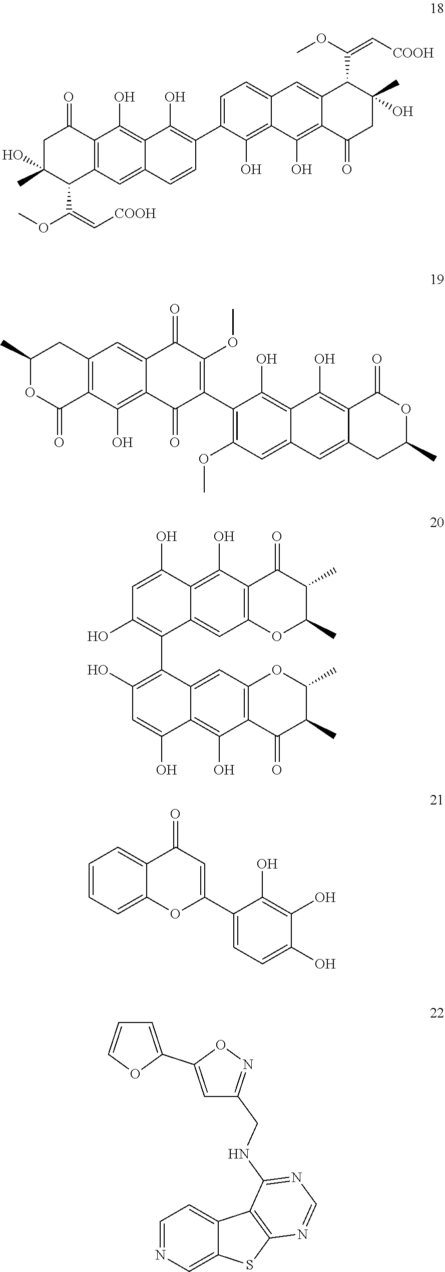

GSK145A (23) was identified as a lead compound that acts as a substrate for Ubc9 and to compete with TRPRS1 binding and SUMOylation. Compounds 24 and 25 are active peptide derivatives.

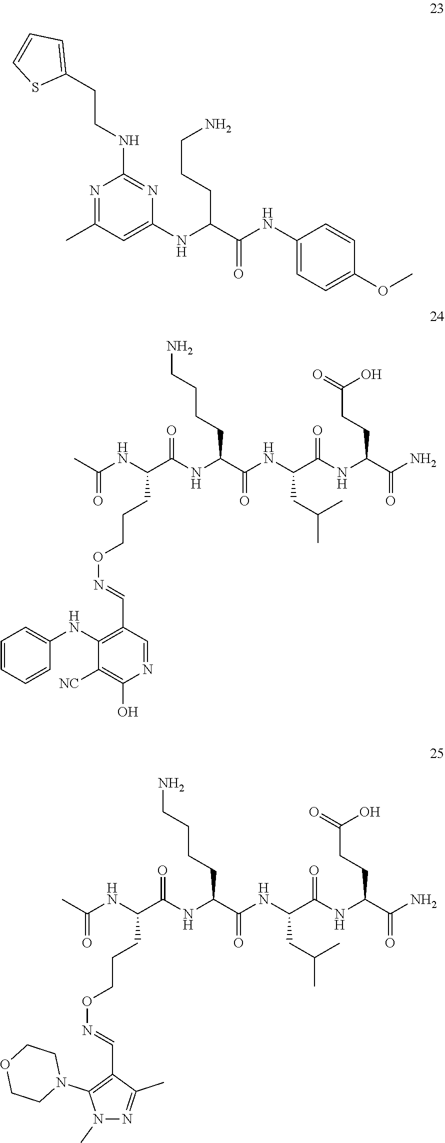 [SMILES: COc1ccc(NC(=O)C(CCCN)Nc2cc(C)nc(NCCc3cccs3)n2)cc1 ; CC(=O)N[C@@H](CCCO/N=C/c1cnc(O)c(C#N)c1Nc1ccccc1)C(=O)N[C@@H](CCCCN)C(=O)N[C@@H](CC(C)C)C(=O)N[C@@H](CCC(=O)O)C(N)=O ; CC(=O)N[C@@H](CCCO/N=C/c1c(C)nn(C)c1N1CCOCC1)C(=O)N[C@@H](CCCCN)C(=O)N[C@@H](CC(C)C)C(=O)N[C@@H](CCC(=O)O)C(N)=O]

Compounds 26 and 27 inhibited formation of the SUMO-E2 complex, but not the SUMO-E1 complex, confirming their compounds bind and selectively inhibit Ubc9.

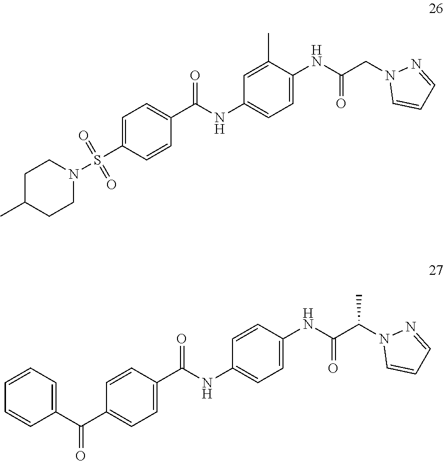 [SMILES: Cc1cc(NC(=O)c2ccc(S(=O)(=O)N3CCC(C)CC3)cc2)ccc1NC(=O)Cn1cccn1 ; C[C@@H](C(=O)Nc1ccc(NC(=O)c2ccc(C(=O)c3ccccc3)cc2)cc1)n1cccn1]

### SENP Inhibitors

Sentrin specific peptidases (SENPs) are responsible for the removal of SUMO from the target protein, with SENP1 and SENP2 responsible for the maturation of SUMO. Like other parts of the SUMOylation pathway, dysregulation of SENPs have been implicated in many cancers.

Several natural products have been identified that inhibit one or more of the SENP isoforms. Momordin Ic (28) was identified from a screen of natural products. Streptonigrin (29) was identified as a potent compound from an iterative screen. Structurally related compounds were also tested, and two analogs that exhibited inhibitory activity, NSC76919 (30) and NSC45384 (31) were identified. Vialinin A (32) and the structurally related natural product, atromentin (33), were evaluated for their ability to inhibit SENP1 in vitro and found that they inhibited SENP1.

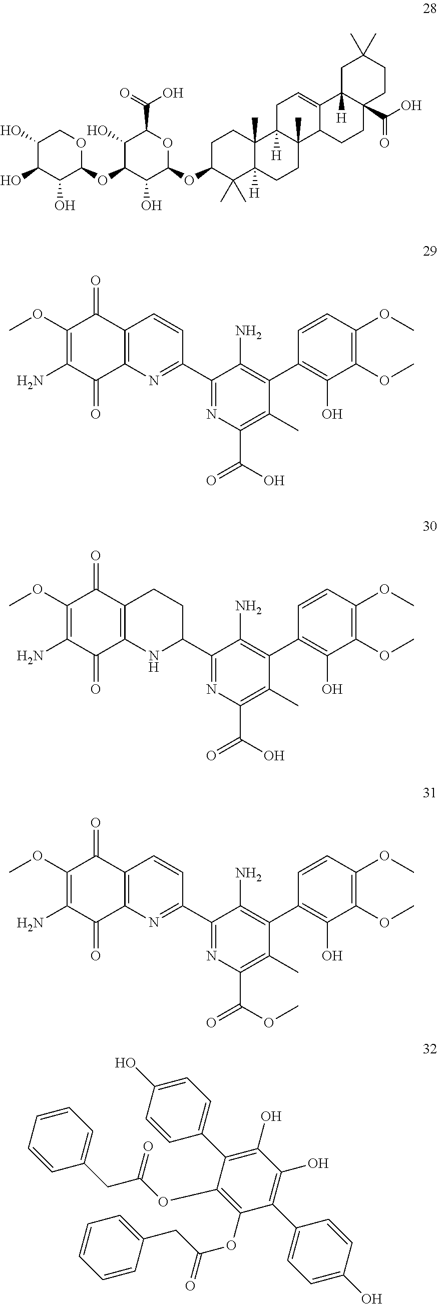

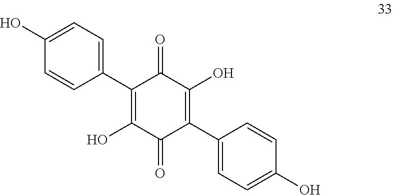 [SMILES: O=C1C(O)=C(c2ccc(O)cc2)C(=O)C(O)=C1c1ccc(O)cc1]

JCP-666 (34) was identified from a screen of cysteine protease inhibitors as an inhibitor of the lone SENP isoform present in Plasmodium falciparum (pfSENP1). A more stable synthetic analog with similar potency, VEA-260 (35), was used developed. Analogs of 35 were developed with more potent and selective SENP inhibitors, VAE-499 (36) (SEQ ID NO: 1), VAE-500 (37) (SEQ ID NO: 2) and VAE-561 (38) (SEQ ID NO: 3).

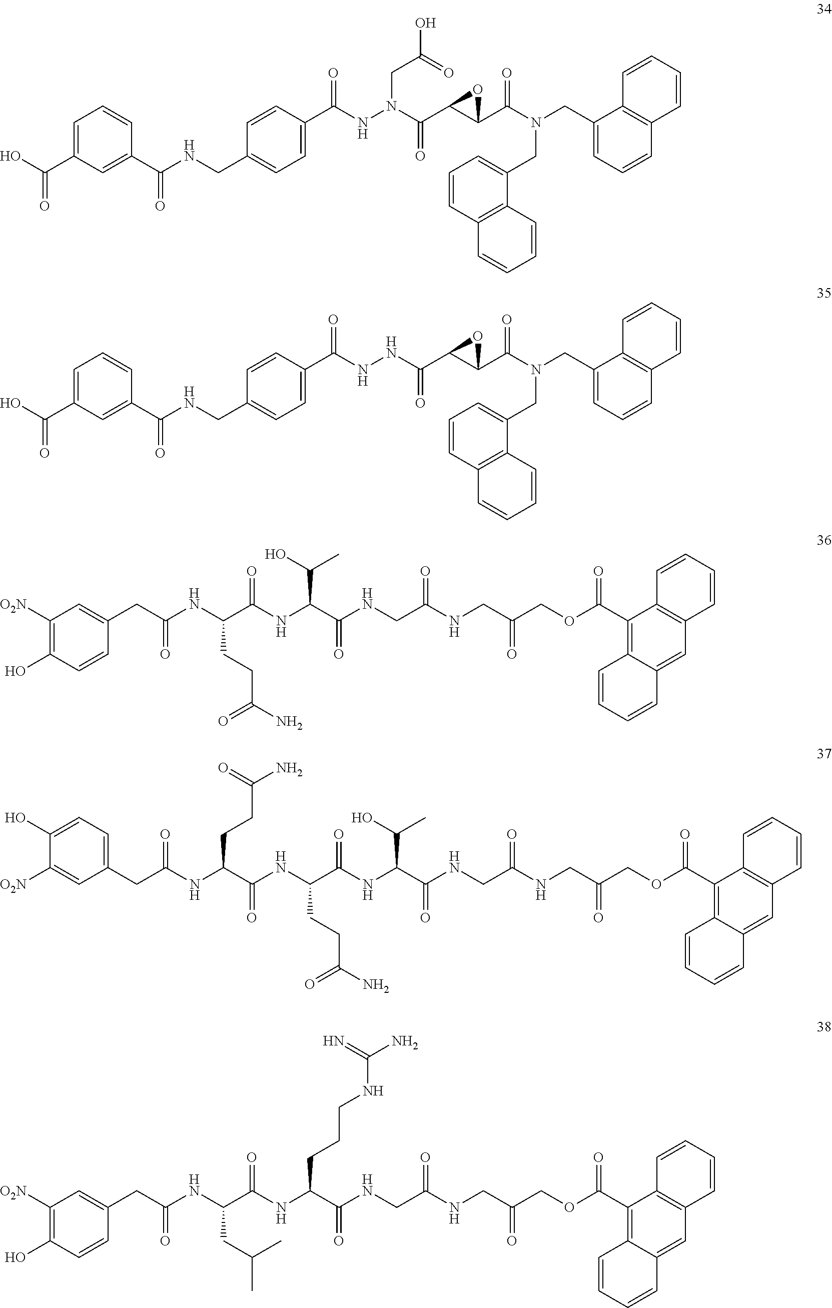

Compound 39, exhibited potent and selective SENP inhibition. GN6767 (40) was able to bind SENP1 in a pull down assay. Subsequent in vitro evaluation confirmed that 40 also inhibited SENP1. Therefore, a small library of analogs was prepared and eventually led to the discovery of GN6958 (41).

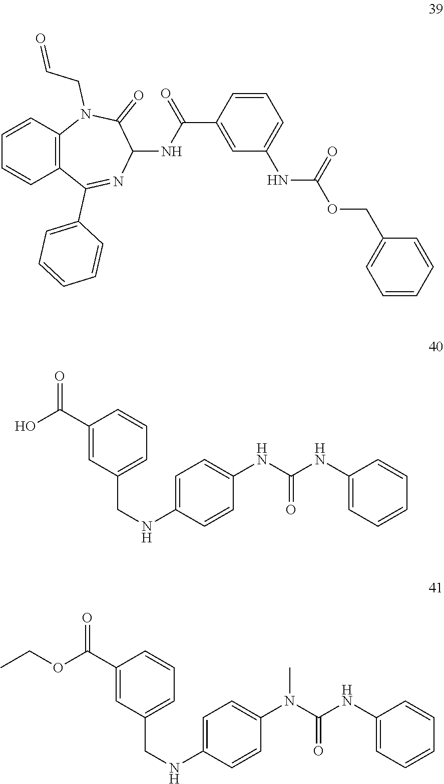 [SMILES: O=CCN1C(=O)C(NC(=O)c2cccc(NC(=O)OCc3ccccc3)c2)N=C(c2ccccc2)c2ccccc21 ; O=C(Nc1ccccc1)Nc1ccc(NCc2cccc(C(=O)O)c2)cc1 ; CCOC(=O)c1cccc(CNc2ccc(N(C)C(=O)Nc3ccccc3)cc2)c1]

Virtual screens have been used to discover several classes of SENP inhibitors, such as compound 42. A small library of derivatives was then synthesized and led to the identification of compound 43, which had a modest improvement in activity. Compounds, SPI-01 (44) and SPI-02 (45), were more active at inhibiting SUMO2 maturation, as well as more active against SENP2. Compounds 46 and 47, and SI2 (48) exhibited potent and selective SENP inhibition. Compounds 49, 50, 51, 52, 53, and 54 exhibited potent and selective SENP inhibition.

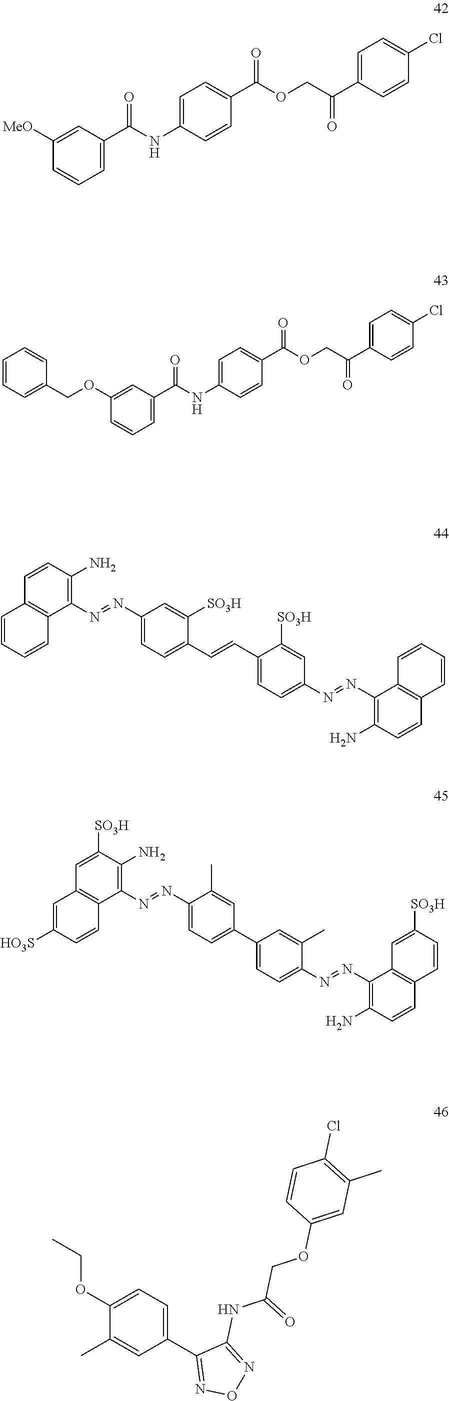 [SMILES: COc1cccc(C(=O)Nc2ccc(C(=O)OCC(=O)c3ccc(Cl)cc3)cc2)c1 ; O=C(COC(=O)c1ccc(NC(=O)c2cccc(OCc3ccccc3)c2)cc1)c1ccc(Cl)cc1 ; Nc1ccc2ccccc2c1/N=N/c1ccc(/C=C/c2ccc(/N=N/c3c(N)ccc4ccccc34)cc2S(=O)(=O)O)c(S(=O)(=O)O)c1 ; Cc1cc(-c2ccc(/N=N/c3c(N)c(S(=O)(=O)O)cc4cc(S(=O)(=O)O)ccc34)c(C)c2)ccc1/N=N/c1c(N)ccc2ccc(S(=O)(=O)O)cc12 ; CCOc1ccc(-c2nonc2NC(=O)COc2ccc(Cl)c(C)c2)cc1C]

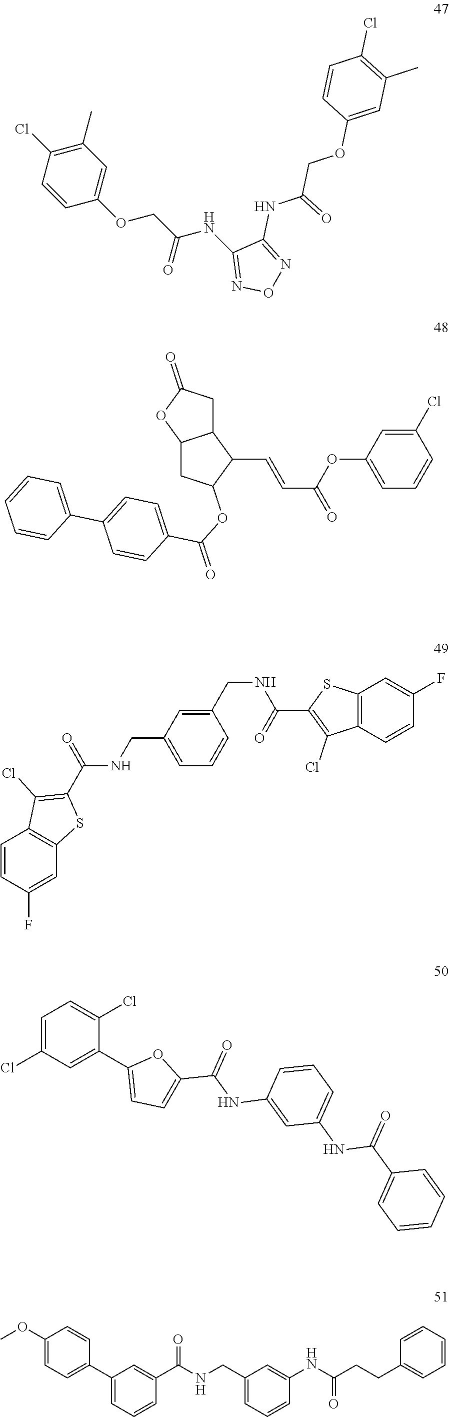

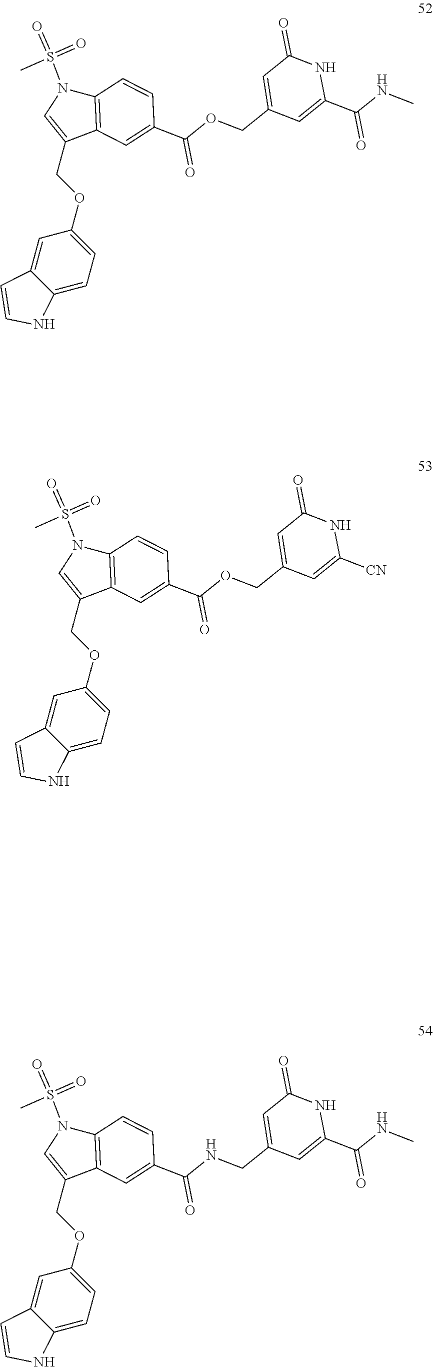 [SMILES: CNC(=O)c1cc(COC(=O)c2ccc3c(c2)c(COc2ccc4[nH]ccc4c2)cn3S(C)(=O)=O)cc(=O)[nH]1 ; CS(=O)(=O)n1cc(COc2ccc3[nH]ccc3c2)c2cc(C(=O)OCc3cc(C#N)[nH]c(=O)c3)ccc21 ; CNC(=O)c1cc(CNC(=O)c2ccc3c(c2)c(COc2ccc4[nH]ccc4c2)cn3S(C)(=O)=O)cc(=O)[nH]1]

### Other SUMOylation Inhibitors

Several molecules have been discovered that modulate the SUMOylation pathway wherein the mode of action is undefined. The first molecule is topotecan (55). Four ortho-quinone natural products have also been identified as potent SUMOylation inhibitors, nocardione A (56), 33-DINOR-dunnione (57), 33-DINOR-dehydrodunnione (58), and β-lapachone (59). A pyrroloiminoquinone, macrophilone A (60), isolated from Macrorhynchia philippina, also demonstrated SUMOylation inhibitory activity through formation of ROS. The methoxy derivative (61) was also synthesized and evaluated and similar to quinones 56-59, iminoquinones 60 and 61 also generated ROS and subsequent E1 and E2 crosslinking. Triptolide (62) decreased SENP1 mRNA levels. Compound N106 (63) is capable of increasing SUMOylation through the activation of SUMO-E1.

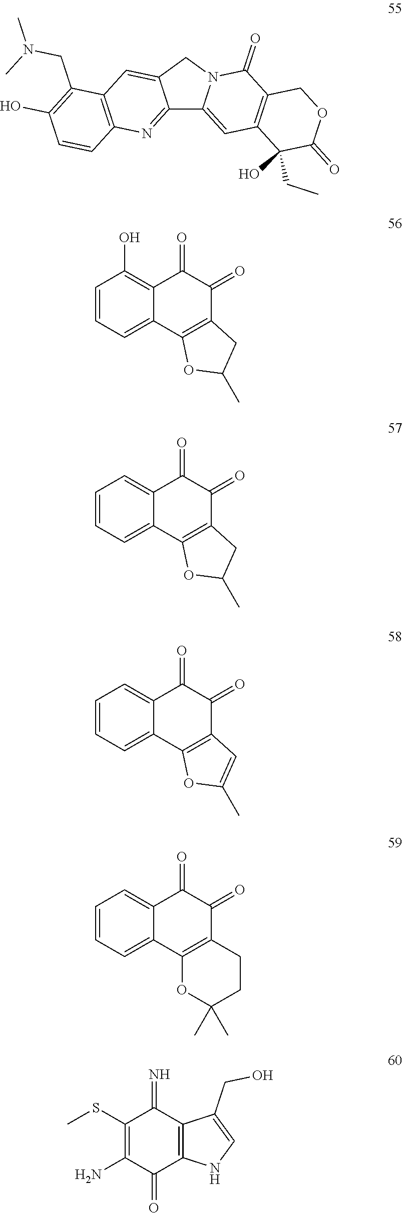 [SMILES: CC[C@@]1(O)C(=O)OCc2c1cc1n(c2=O)Cc2cc3c(CN(C)C)c(O)ccc3nc2-1 ; CC1CC2=C(O1)c1cccc(O)c1C(=O)C2=O ; CC1CC2=C(O1)c1ccccc1C(=O)C2=O ; Cc1cc2c(o1)-c1ccccc1C(=O)C2=O ; CC1(C)CCC2=C(O1)c1ccccc1C(=O)C2=O ; CSC1=C(N)C(=O)c2[nH]cc(CO)c2C1=N]

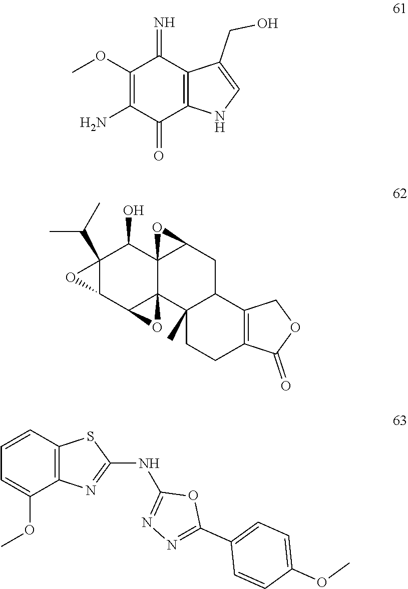 [SMILES: COC1=C(N)C(=O)c2[nH]cc(CO)c2C1=N ; CC(C)[C@]12O[C@H]1[C@@H]1O[C@]13[C@]1(O[C@H]1CC1C4=C(CC[C@@]13C)C(=O)OC4)[C@@H]2O ; COc1ccc(-c2nnc(Nc3nc4c(OC)cccc4s3)o2)cc1]

Disclosed are methods of treating or preventing a cardiovascular disease. The method may comprise administering to a subject in need thereof an effective amount of a compound. The compound may be a SUMOylation inhibitor, an analogue of Phosphatidylinositol 4,5-bisphosphate (PIP2); PIP2; or a compound that increases endogenous PIP2.

In some embodiments, the compound is a SUMOylation inhibitor; and the compound is topotecan, nocardione A, 33-DINOR-dunnione, 33-DINOR-dehydrodunnione, and β-lapachone, macrophilone A, compound 61, Triptolide, or N106. In some embodiments, the compound is a SUMOylation inhibitor; and the compound is an inhibitor of SAE1. In some embodiments, the inhibitor of SAE1 is ginkgolic acid, anacardic acid, Kerriamycin B, Davidiin, tannic acid, SUMO-AMSN, SUMO-AVSN, phenyl urea compounds, compound 10, pyrazole urea, thiazole urea, CID9549553, COH000, compound 15, ML-792, pevonedistat, or TAK-981. In some embodiments, the compound is a SUMOylation inhibitor; and the compound is an inhibitor of SAE2. In some embodiments, the inhibitor of SAE2 is spectomycin B1, chaetochromin A, viomellein, flavone 2-D08, compound 22, GSK145A, compound 24, compound 25, compound 26, or compound 27. In some embodiments, the compound is a SUMOylation inhibitor; and the compound is an inhibitor of SENP1. In some embodiments, the inhibitor of SENP1 is momordin Ic, streptonigrin, NSC76919, NSC45384, vialinin A, atromentin, JCP-666, VEA-260, VAE-499, VAE-500, VAE-561, compound 39, GN6767, GN6958, compound 42, compound 43, SPI-01, SPI-02, compound 46, compound 47, SI2, compound 49, compound 50, or compound 51. In some embodiments, the compound is a SUMOylation inhibitor; and the compound is an activator of SENP1.

In some embodiments, the compound increases endogenous PIP2; and the compound is an inhibitor of a Gαq-coupled AT1 receptor. In some embodiments, the inhibitor of a Gαq-coupled AT1 receptor is losartan, Exp 3174, telmisartan, irbesartan, candesartan, valsartan, eprosartan, azilsartan, saprisartan or olmesartan. In some embodiments, the compound increases endogenous PIP2; and the compound is an inhibitor of phospholipase C (PLCβ). In some embodiments, the inhibitor of PLCβ is U73122, phenylmethylsulfonyl fluoride, manoalide, D609, ET-18-OCH3, compound 48/80 trihydrochloride, spermine tetrahydrochloride, neomycin sulfate, NCDC, or thielavin B. In some embodiments, the compound is an analogue of PIP2; and the PIP2 analogue is diC8-PIP2. In some embodiments, the compound increases endogenous PIP2; and the compound inhibits PIP2 hydrolysis or inhibits PIP2 dephosphorylation.

In some embodiments, the cardiovascular disease is a hypertension, arrhythmia, coronary heart disease, cerebrovascular disease, rheumatic heart disease, ventricular hypertrophy, heart failure, vasculitis, atherosclerosis, myocardial infarction, angina pectoris, renal failure, transient ischemic attacks, peripheral vascular disease, aneurysm formation, hypercholesterolemia, hyperlipidemia, hyperlipoproteinemia, deep vein thrombosis, ventricular arrythmia, supraventricular tachycardia, or platelet aggregation. In some embodiments, the hypertension is pulmonary artery hypertension, systemic hypertension, pulmonary hypertension, sporadic pulmonary arterial hypertension, familial pulmonary arterial hypertension, idiopathic pulmonary arterial hypertension, or acquired pulmonary arterial hypertension. In some embodiments, the patient has a disease that reduces blood oxygen levels. In some embodiments, the patient has an infection, inflammation, sepsis, cystic fibrosis, COPD, sleep apnea, or cancer.

### EXAMPLES

The invention now being generally described, it will be more readily understood by reference to the following examples, which are included merely for purposes of illustration of certain aspects and embodiments of the present invention, and are not intended to limit the invention.

### Example 1: Hypoxia-Induced SUMOylation in Human PASMCs

Our data show hypoxia-induced SUMO-regulation of Kir2 channels. Further, these effects are precluded when cells are pretreated with TAK981. Using single-cell sequencing technology we identify gene expression patterns across PASMC populations and assess changes to expression following hypoxic insults. The interaction of candidate channels with SUMO is confirmed using proximity ligation assay (PLA), Western blots, and shRNA knockdown to assess their functional role in PASMCs. Relevant channels are assessed in electrophysiology experiments to establish sensitivity to TAK981.

Hypoxic-inhibition of Kir2.1 in cardiomyocytes via SUMOylation. We studied the effects of acute hypoxia on I_{K1} in rat ventricular cardiomyocytes (RVCMs) by whole cell patch-clamp recording. Reducing the O₂-tension of the superfusate from ambient levels to 2% O₂ resulted in a rapid 40±0.1% (mean±S.D.) decrease in I_{K1} of over 90 seconds (FIG. 1a). Both the hypoxia-sensitive and insensitive components of the current were blocked by Ba²⁺ (3 mM), consistent with the known role of Kir2 channels in mediating I_{K1} (FIG. 1a). To determine if SUMOylation mediates the hypoxic inhibition of I_{K1} we introduced purified SENP1 (2 μM) to the cytosol via the recording pipette. SENP1 increased I_{K1} by 17.6±6.5%, indicating that the current was partially inhibited by SUMOylation under resting conditions, as observed with other membrane localized SUMO-target proteins. Furthermore, treatment with SENP1 rendered I_{K1} insensitive to hypoxia (FIGS. 1a, 1b). In contrast, when we included SUMO1 (1 μM) in the recording pipette (a maneuver that increases SUMO-conjugation to ion channels), I_{K1} decreased by 48±8% and the hypoxia-sensitive component of the current was abolished (FIG. 1b). Both the SUMO1 and hypoxia-mediated inhibition of I_{K1} remained stable throughout the remainder of the experiment (up to 10 mins). Together, these results suggest that SUMOylation mediates the hypoxia-induced inhibition of I_{K1} in cardiomyocytes. Next, we visualized and quantified the interactions between native Kir2.1 and SUMO1 in RVCMs using the proximity-ligation association (PLA) assay with antibodies we validated in heterologous cells by Western blot. Because our functional studies predict that only up to ˜20% of Kir2.1 channels are SUMOylated at baseline we did not anticipate visualizing the small population of SUMO-adducts under these experimental conditions. The validated Kir2 antibodies were paired with SUMO1 antibodies that we have tested and utilized before. Under control conditions, interactions were observed between Kir2.1 and SUMO1, but not between Kir2.2 or Kir2.3 and SUMO1. Exposing RVCMs to acute hypoxia resulted in a significant increase in the number of interactions between native Kir2.1 and SUMO1 consistent with the conclusion that hypoxia-augments the SUMOylation of Kir2.1 channels in cardiomyocytes (FIG. 1c). Although cardiomyocytes are typically cultured at 21% O₂, the O₂-level in the myocardium is estimated to be in the range of 5-10% O₂, under physiological conditions. Culturing RVCMs at 7% O₂ had no effect on the capacity for acute hypoxia (2% O₂) to rapidly diminish the current, and did not reduce the frequency of positive PLA interactions between Kir2.1 and SUMO1.

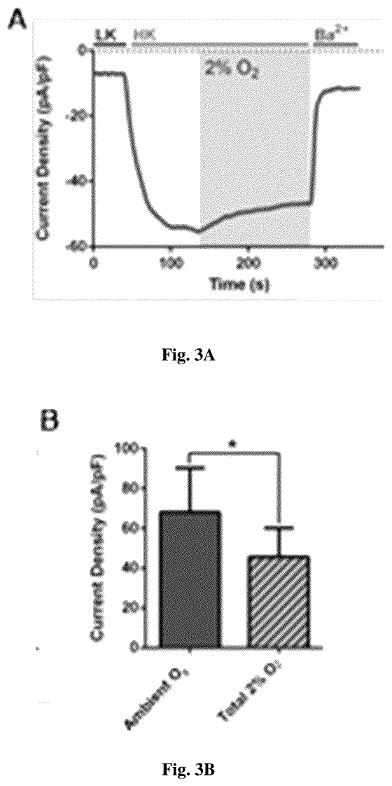

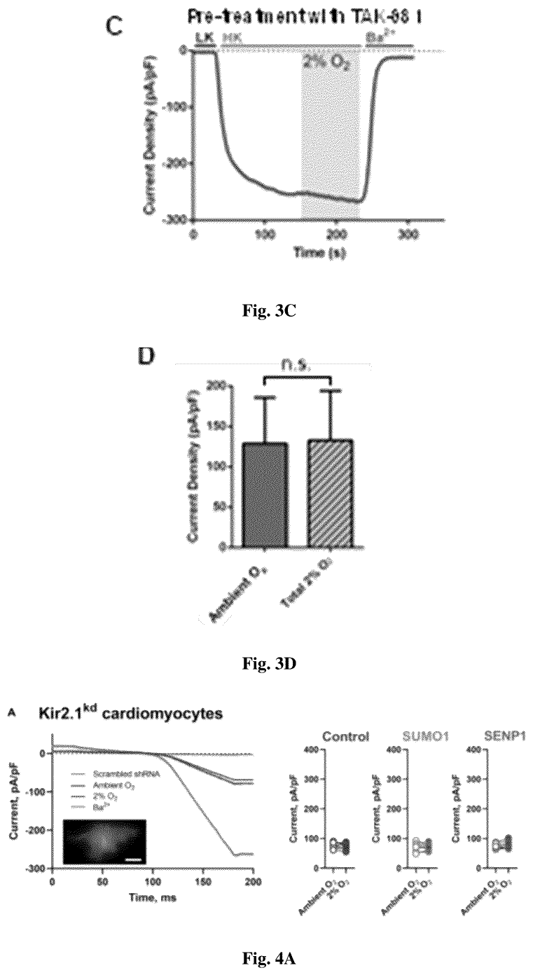

Hypoxia diminishes Kir2.4 activity in a TAK981 sensitive manner. HEK293T cells were transiently transfected with plasmid DNA for Kir2.4 channels. Inward rectifying currents were elicited in response to a high [K]⁺ (140 mM) buffer using a −80 mV to +80 mV ramp protocol. Following stabilization in the high K⁺ buffer, the cells were subjected to a hypoxic (2% O₂) perfusate. Acute hypoxia evoked a ˜30% decrease in Kir2.4 currents. Recordings were terminated with the perfusion of 10 mM Ba²⁺ (FIGS. 3A, 3B).

Next, we repeated the experiment using that were preincubated with 100 nM TAK981 for one hour. We observed that the hypoxic decrease in Kir2.4 currents was abolished in cells pre-treated with TAK981 (FIGS. 3C, 3D). These results suggest that, in-line with our work on Kir2.1, hypoxia mediates its effects on Kir2.4 involves activation of the SUMO pathway (FIG. 2).

### Experimental Design

Hypoxia-induced changes in K⁺ channel expression patterns. We have extensively studied the SUMOylation of ion channels in hypoxia. Numerous hypoxia and SUMO sensitive channels are implicated in the physiology in PASMCs. These studies have employed cells from a range of species with inherently different HPV responses. We assess whether acute hypoxia (˜3 min at 2% O₂) or chronic hypoxia (12 h) changes the expression pattern of channels in human PASMCs. To accomplish this, we culture human primary PASMCs from Lonza to meet the experimental conditions. For acute hypoxia, we culture the cells in a normoxic or 7% O₂ incubator and then acutely expose the cells to 2% O₂ using a perfusion system from our electrophysiology experiments. The cells are perfused with a physiological buffer that had been bubbled with nitrogen for 30 minutes prior to the experiment. O₂ tension is measured at the cell using a calibrated O₂ probe (Ocean Insight) to deliver ˜2% O₂ in the sample chamber. Then, we use 10× Genomics single cell sequencing technology through the Next Generation Sequencing Core Facility to characterize the expression pattern of ion channels in PASMC populations under control and hypoxic conditions. This experiment provides a library of targets at the genetic level. We compare these findings with those in those in the literature, for example the RT-PCR identification of robust Kir2.4 expression in human PASMCs.

In parallel, we fix a portion of each cell sample and use PLA (FIG. 1) to classify baseline and hypoxic induced interactions with SUMO (FIG. 1). PLA is performed on cells seeded to glass coverslips and perfused with 21% or 2% O₂ solution for 3 mins then fixed in 4% paraformaldehyde. Kir2 and SUMO isoforms are detected by incubating with 1° antibodies (Kir2.1 mouse monoclonal from Neuromab; SUMO1 rabbit monoclonal from AbCam, clone Y299) at 4° C. overnight. PLA signals are detected and amplified with a commercial kit (Sigma Aldrich) and visualized with a Zeiss LSM880 confocal microscope. Nuclei are identified with DAPI.

SUMOylation as an O₂-sensor in PASMCs. Using patch-clamp, we perform a series of initial experiments to show that SUMO pathway activity is increased in acute hypoxia, leading to depolarization of the cells, as we found in cerebellar granule neurons, iPS-derive human cardiomyocytes, and rat ventricular cardiomyocytes. First, we study the cell in both voltage-clamp and current-clamp modes to assess changes in membrane potential and membrane current in response to acute hypoxia (2% O₂). To induce acute hypoxia, cells are perfused with a recording buffer that has been bubbled with nitrogen for 30 minutes prior to recording. O₂ tension was measured at the cell using a calibrated O₂ probe (Ocean Insight) to confirm ˜2% O₂ in the recording chamber. Next, we use a series of voltage-protocols to capture Kir, K2P and Kv currents in the cells and study the effect of acute hypoxia on the PASMCs. Based on these findings, we determine the role of SUMOylation in the acute hypoxic inhibition of the currents. We promote SUMOylation by adding purified SUMO1 protein to the patch pipette. Based on our studies and data, SUMO1 decreases the current through SUMO-sensitive channels, identifying which components of the overall K⁺ currents are subject to this regulation. In contrast, the deSUMOylating enzyme SENP1 opposes the effects of hypoxia. Observing that SENP1 precludes hypoxia-induced changes in some, or all components of the K⁺ current in the PASMCs, suggest that the SUMO pathway is an O₂-sensor in these cells.

The effect of a SUMO inhibitor on PASMCs. In addition to purified SUMO1 and SENP1, we test the role of the SUMOylation pathway in PASMCs O₂-sensing using TAK981. Our data show that pretreating cells with TAK981 is tolerated by the cells and precludes hypoxic inhibition of Kir2 currents in HEK293 cells (FIG. 3) and RVCMs. Because most of the SUMO is conjugated to E1, E2 (Ubc9) or target proteins cells must be preincubated with TAK981 (Cayman Chemical) prior to assays, in order to observe maximal efficacy (FIG. 2). Here, we preincubate PASMCs prior to patch-clamp studies. Based on our findings, we preincubate the cells with 100 nM TAK981 for 60-90 mins. We, however, perform concentration-response studies with our own hands to optimize the concentration for use in PASMCs. We again record currents that we observe to be SUMO and hypoxia sensitive and test if the effects of hypoxia are precluded, as we observe in our data (FIG. 3). We expect that TAK981 will preclude the pro-SUMOylating effects of hypoxia on at least some K⁺ currents. To test if the effects of TAK981 are sufficient to preclude our proposed mechanism on the physiology of PASMCs, we also perform current-clamp experiments to study hypoxia-induced depolarization of the cells.

The SUMO pathway inhibitor TAK981 prevents hypoxia induced changes ion channel current sin human pulmonary artery smooth muscle cells (hPASCMs) (FIGS. 10A-10B). The hPASMCs are in primary culture and were studies by whole cell patch clamp at ambient levels of oxygen (˜21% O₂) and under acute hypoxia (2% O₂). Cells were studied in quasi-physiological buffers with high external concentrations of potassium using recording protocols.

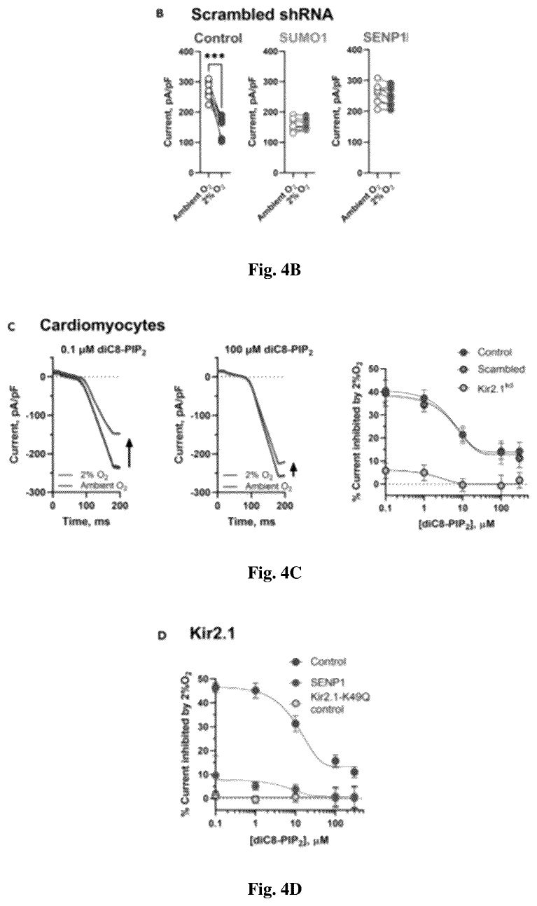

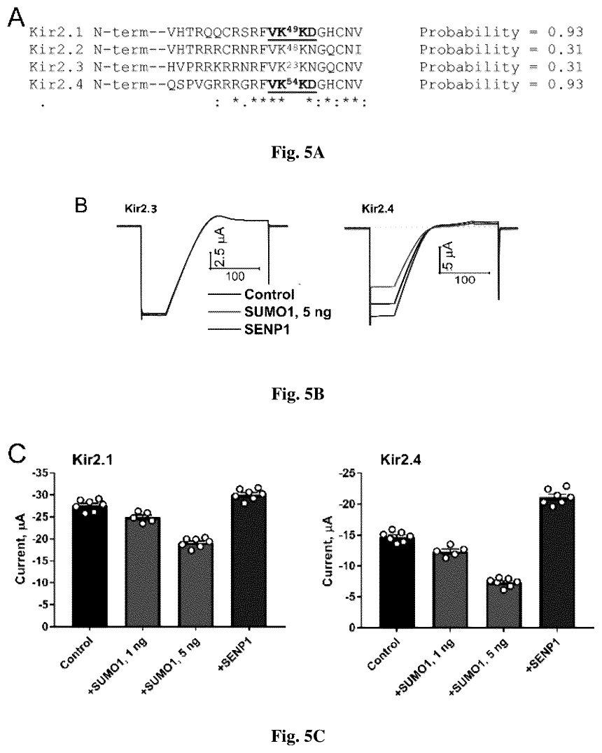

The effects of specific channels on PASMCs O₂-induced depolarization. Based on the results of the 10× genomics screen, and the PLA study, we identify prominently expressed channels that participate in the physiology of PASMCs. Several of these candidates are K_{V}1.5, K_{V}2.1, K2P3, Kir2.4, and others. To confirm a functional role for these channels in the depolarization of PASMCs, we knockdown genes of interest using targeted shRNA, as shown in our data (FIG. 4). The shRNA construct is purchased in a lentiviral vector (Origene) and the efficiency of mRNA knockdown is assessed based on (1) correlated GFP expression from the bicistronic lentiviral vector (FIG. 4A) and (2) single-cell PCR. In all cases, a scrambled sequence shRNA is used in parallel to control for the viral transduction. If shRNA is inefficient, then we use CRISPR to knockout the gene of interest. Similar studies are performed with hypoxia, SUMO1 and SENP1 in the knockdown cells. In our studies, this approach knocked down >90% of the Kir2.1 current in RVCMs, rendering the I_{K1} current smaller, but resistant to hypoxic-inhibition (FIGS. 4A-4B).

### Example 2: SUMO Modulates Kir2.4 Channels Via PIP2

We characterize the SUMO-regulation of Kir2.4 and the role of PIP2 in mediating effects on channel function. Using site-directed mutagenesis, we probe the SUMO motif on Kir2.4 in electrophysiology experiments and FRET in tandem to determine whether abolishing the SUMO site alters FRET association or electrophysiology changes. This study is extended to other channel targets from Example 1.

Overview. Here, we focus on SUMO-regulation of Kir2.4, an understudied K⁺ channel in PASMCs. Our data show that this channel is both hypoxia and SUMO-sensitive and that the hypoxic regulation can be ameliorated by pretreating the cells with TAK981. Further, we show that the SUMO-sensitivity of this channel requires the membrane phospholipid PIP2, which is essential for channel gating.

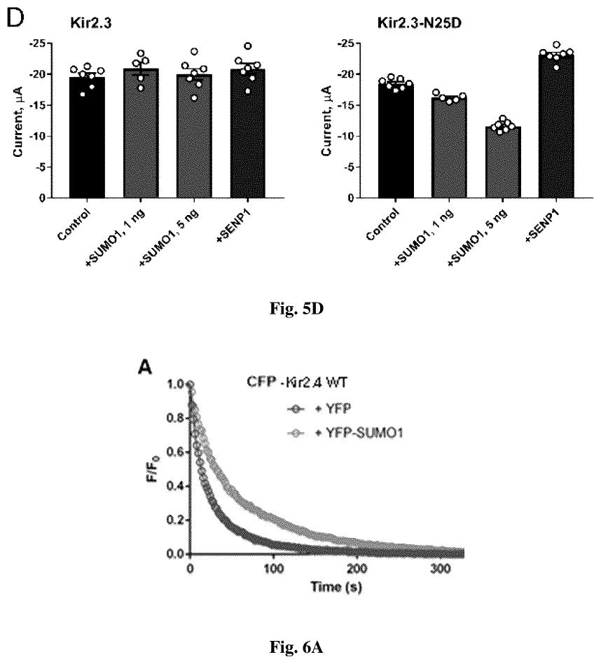

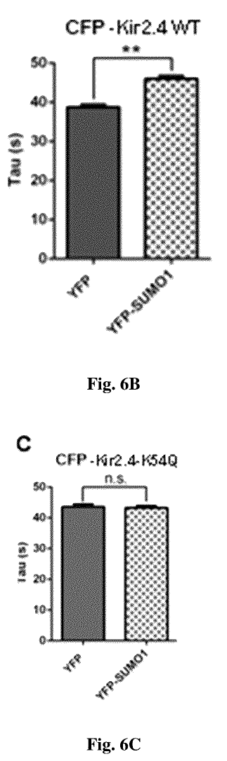

Co-expressed SUMO decreases Kir2.4 currents in Xenopus oocytes. Using the SUMOplot algorithm to assess potential SUMOylation sites based on the canonical motif ψ-K-X-E/D, we identified the high probability SUMO site V-K₅₄-K-D (SEQ ID NO: 4) in the N-terminal sequence of Kir2.4. SUMO covalently modifies Kir2.4 at K₅₄ (FIG. 5) and tested the idea using two-electrode voltage-clamp (TEVC), a sensitive assay to study the regulation of Kir channels. Kir2.4 currents, assessed at −80 mV in a high K⁺ (96 mM) bath solution, decreased in a dose-dependent manner with co-expression of SUMO1 (FIG. 5). Co-injection of 5 ng of human SUMO1 RNA decreased Kir2.4 currents by ˜50%. In contrast, expression of SENP1 increased Kir2.4 by ˜50%. The action of SENP1 suggested that (1) the channels are partially SUMOylated at baseline and (2) the SUMOylation is covalent (FIG. 5). The analogous sequence in Kir2.2 and Kir2.3 is V-K-K-N(SEQ ID NO: 5), which lacks the terminal aspartic acid, reduced the probability of SUMOylation from 0.93 to 0.31. Consistent with this, Kir2.3 currents were insensitive to SUMO1 and SENP1. However, SUMO-regulation was transferred to Kir2.3 by the mutation N25D, recreating the V-K-K-D motif (SEQ ID NO: 4) from Kir2.1/2.4 (FIG. 5), suggesting that the mechanistic basis for channel SUMOylation is common in the Kir2s studied. Upon mutation of the predicted lysine residue, K54 to Q in Kir2.4, we observed an absence of association between YFP-SUMO and mTFP-Kir2.4 in donor-decay FRET experiments, indicating that K54 is critical to the SUMOylation of Kir2.4 (FIG. 6). This last result is consistent with a protein-protein interaction between the channel and SUMO1.

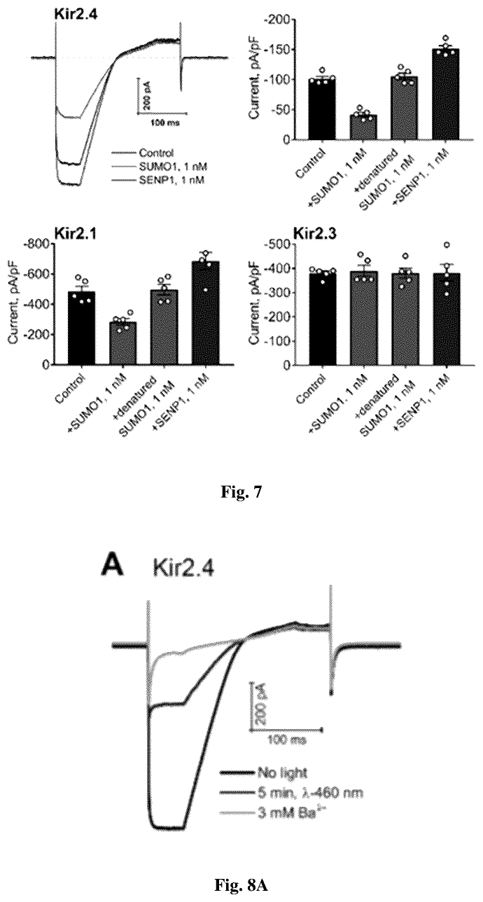

SUMOylation regulates Kir2.4 channels in mammalian cells. Next, we tested if SUMO could regulate Kir2.4 channels in mammalian cells. Kir2.4 channels were transiently expressed in HEK293 cells and studied in whole-cell mode with 140 mM bath [K]⁺, using a ramp protocol from −80 to +80 mV. Purified SUMO1 or SENP1 peptide were introduced via the recording pipette. At 1 nM, SUMO1, but not heat-denatured SUMO1, inhibited Kir2.4 currents by ˜40 and 60%, respectively, within one-minute of breakthrough. In contrast, 1 nM SENP1 increased the current by ˜50% (FIG. 7).

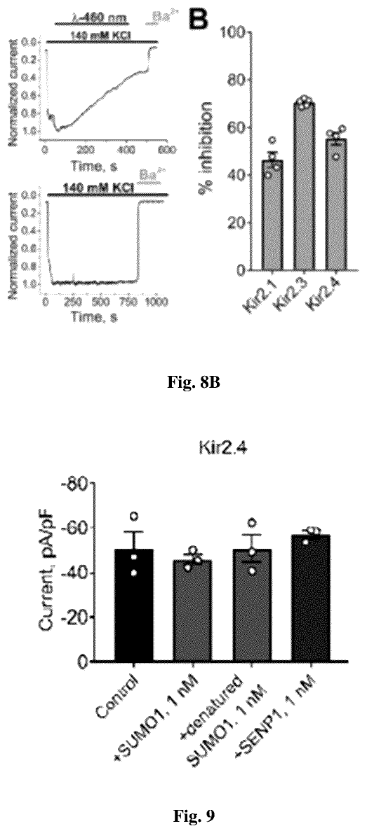

Optogenetic control of PIP2 levels in HEK cells. Many Kir2 regulators operate by interfering with the interaction between the channel and PIP2. Thus, regulators consistently exert a greater effect on Kir2.2, 2.3 and 2.4 than on Kir2.1, because the apparent affinity for PIP2 is greatest for the latter. Three lines of evidence suggested that SUMO-regulation is PIP2-dependent: (1) SUMOylation decreased Kir2.4 currents more than Kir2.1 currents; (2) the SUMO-motifs coincide with sites known to co-ordinate PIP2 and (3) SUMO-sensitivity could be transferred to Kir2.3 via a point mutation. To further test this idea, we depleted PIP2 at the membrane of HEK293 cells with the light-activated, membrane-associated 5-phosphatase system: mCherry-CRY2-5-ptasc_{OCRL}/CIBN-CAAX (5-ptase_{OCRL}). 5-ptase_{OCRL} has been extensively used to precisely dephosphorylate PIP₂ with high spatiotemporal accuracy. Activation of 5-ptasc_{OCRL}, by 460 nm light decreased currents through Kir2.4 by ˜55%. (FIG. 8). This result confirmed activation of 5-ptasc_{OCRL}, as a viable strategy to study the PIP2-dependence of Kir2 channels.

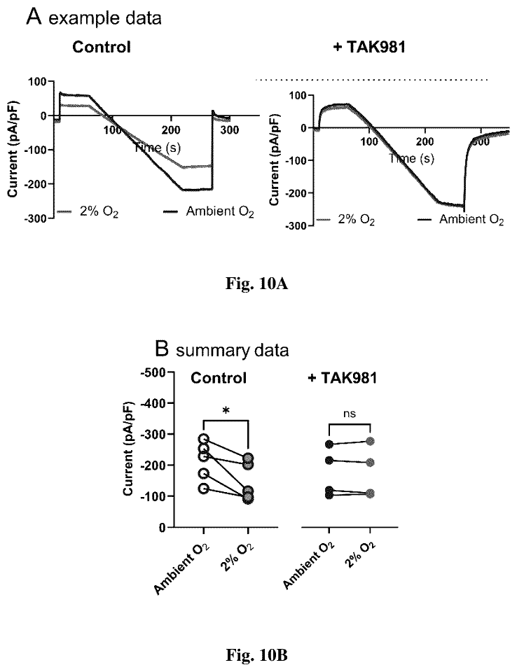

The PIP2-dependence of SUMO-regulation. Using the 5-ptase_{OCRL} system, we depleted PIP2 in HEK293 cells expressing Kir2.4 and studied currents with 1 nM SUMO1, or SENP1 in the recording pipette. When currents were recorded after 5-min activation of the optogenetic probe, 5-ptase_{OCRL}, the inhibitory effect of 1 nM SUMO1 on Kir2.4 was reduced from ˜40% to ˜7% (FIG. 9). Current augmentation observed with SENP1 in the pipette was reduced from ˜40% to ˜6%. These preliminary data support the conclusion that SUMO-regulation of Kir2.4 channels is PIP2-dependent.

Hypoxic-inhibition of I_{K1}/Kir2.1 is reduced by diC8-PIP2 in a concentration-dependent manner. We have shown that adding the exogenous, soluble PIP2 analog diC8-PIP2 into the patch-pipette opposes hypoxic-inhibition of I_{K1} (FIG. 4). Including diC8-PIP2 in the pipette diminished the hypoxic-inhibition of I_{K1} in a concentration-dependent manner in wild-type RVCMs, with 100 μM diC8-PIP2 reducing hypoxic inhibition of I_{K1} from ˜40% to ˜15%. The same result was observed in RVCMs transduced with the scrambled shRNA but was abolished in cells where the expression of Kir2.1 was knockdown by a lentiviral shRNA targeting kcnj2 (Kir2.1kd-CMs FIG. 4C). Adding diC8-PIP2 in the recording pipette also produced a concentration-dependent reduction in hypoxic inhibition of heterologous Kir2.1 channels expressed in HEK293T cells. As before, the effects of hypoxia were ameliorated by SENP1 (2 μM) and were not observed in the SUMO and hypoxia insensitive Kir2.1-K49Q channels (FIG. 4D).

### Experimental Design

Our results suggest that Kir2.4 channels are SUMO-regulated, and that SUMO-regulation is weaker when PIP2 is depleted. Together, these findings give rise to the conclusion that SUMO-regulation of Kir2.4 channels occurs because SUMO modulates the channel-PIP2 interaction. The role of this regulation in PASMC function and its molecular mechanism is unknown. Here, we describe an experimental strategy to characterize SUMO-regulation of Kir2.4 in PASMCs and heterologous cells.

Characterize SUMO-regulation of Kir2.4. Wild type and lysine mutant Kir2.4 channels are studied in mammalian cells (HEK293) by whole-cell patch-clamp recording. To study channels that are not tagged with a fluorescent protein (FP), eGFP is used as a transfection marker. Currents are studied in a 140 mM K⁺ bath solution, using the same recording protocols described above, with 3 different pipette solutions: a control solution, a solution with SUMO1, or SENP1. SUMO1 and SENP1 are purchased from Boston Biochem (RnD systems) and are used at a range of concentrations to establish the dose-dependency of the regulation. We also study the effect of SUMO2 and SUMO3 peptides on the channels. Kir2.4-K54Q channels are not expected to respond to SUMO1 or SENP1 and provide a baseline measurement for the deSUMOylated state. Similarly, our work predicts that Kir2.1 and Kir2.4 are SUMOylated at a single lysine residue. Our studies show that this assertion can be tested by live cell FRET and TIRFM. A previously characterized SUMO1 target (such as CFP-Kv2.1) is used as a positive control in these experiments and both free YFP, and the covalently incapable SUMO mutant, SUMO195, act as a negative control. FRET also allows assessment of the interaction between Kir2 channels and SUMO2 and SUMO3, as well as other potential protein partners, including the SUMO pathway enzymes that we have observed in complex with K⁺ channels. In principle, SUMOylation at other sites might not have an electrophysiological impact on the Kir2 channel. Alternatively, the fluorescent protein-tag might promote or hinder channel function/SUMOylation. To control for this possibility, the constructs used in microscopy studies are tested by whole-cell patch-clamp.

The PIP2-dependence of Kir2-SUMO regulation. Having established conditions that generate maximal SUMO-regulation of Kir2.4 channels in oocytes and tissue culture cells, we take advantage of well characterized assays to investigate the PIP2-dependence of the effect. Since both SUMOylation and PIP2-depletion decrease Kir2.4 currents, we propose that SUMO reduces the currents by interfering with the channel-PIP2 interaction. We test the role of PIP2 in the SUMO-regulation of whole-cell Kir2 channel currents in HEK293 cells, using the 5-ptasc_{OCRL}, system (FIG. 8). While this optogenetic approach avoids drug treatments that might have long-term regulatory effects that impact the experiment, it is important to correlate the degree of PIP2-depletion with the time course of 5-ptasc_{OCRL}, activation in our hands. To accomplish this, we co-transfect the near-red fluorescent PIP2 biosensor iRFP-PHPL.C81. This fluorophore has spectral properties that are distinct from 5-ptasc_{OCRL}, and is compatible with our microscope system. SUMOylated Kir2.4 channels display a larger decrease in current and faster kinetics of inhibition following optogenetic activation of 5-ptasc_{OCRL}. Conversely, wild type Kir2.4 and Kir2.4-K54Q have comparatively slower but equivalent rates and magnitudes of current inhibition. We also study the effect of enhanced PIP2 in HEK293 cells, by increasing levels of PIP2 by application of the water soluble PIP2 analog diC8 in the patch-clamp pipette (FIG. 4). We have shown that this experiment opposes the effects of acute hypoxia on I_{K1} current in RVCMs and Kir2.4 heterologous expressed in HEK293T cells, in a concentration dependent manner (FIG. 4).

### INCORPORATION BY REFERENCE

All publications, US patents, and US and PCT published patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of treating or preventing a cardiovascular disease, comprising administering to a subject in need thereof an effective amount of a compound;
wherein the compound is a SUMOylation inhibitor;
the compound is an analogue of Phosphatidylinositol 4,5-bisphosphate (PIP2);
the compound is PIP2; or
the compound increases endogenous PIP2.

2. The method of claim 1, wherein the compound is a SUMOylation inhibitor; and the compound is topotecan, nocardione A, 33-DINOR-dunnione, 33-DINOR-dehydrodunnione, and β-lapachone, macrophilone A, compound 61, Triptolide, or N106.

3. The method of claim 1, wherein the compound is a SUMOylation inhibitor; and the compound is an inhibitor of SAE1.

4. The method of claim 3, wherein the inhibitor of SAE1 is ginkgolic acid, anacardic acid, Kerriamycin B, Davidiin, tannic acid, SUMO-AMSN, SUMO-AVSN, phenyl urea compounds, compound 10, pyrazole urea, thiazole urea, CID9549553, COH000, compound 15, ML-792, pevonedistat, or TAK-981.

5. The method of claim 1, wherein the compound is a SUMOylation inhibitor; and the compound is an inhibitor of SAE2.

6. The method of claim 5, wherein the inhibitor of SAE2 is spectomycin B1, chaetochromin A, viomellein, flavone 2-D08, compound 22, GSK145A, compound 24, compound 25, compound 26, or compound 27.

7. The method of claim 1, wherein the compound is a SUMOylation inhibitor; and the compound is an inhibitor of SENP1.

8. The method of claim 7, wherein the inhibitor of SENP1 is momordin Ic, streptonigrin, NSC76919, NSC45384, vialinin A, atromentin, JCP-666, VEA-260, VAE-499, VAE-500, VAE-561, compound 39, GN6767, GN6958, compound 42, compound 43, SPI-01, SPI-02, compound 46, compound 47, SI2, compound 49, compound 50, or compound 51.

9. The method of claim 1, wherein the compound is a SUMOylation inhibitor; and the compound is an activator of SENP1.

10. The method of claim 1, wherein the compound increases endogenous PIP2; and the compound is an inhibitor of a Gαq-coupled AT1 receptor.

11. The method of claim 10, wherein the inhibitor of a Gαq-coupled AT1 receptor is losartan, Exp 3174, telmisartan, irbesartan, candesartan, valsartan, eprosartan, azilsartan, saprisartan or olmesartan.

12. The method of claim 1, wherein the compound increases endogenous PIP2; and the compound is an inhibitor of phospholipase C (PLCβ).

13. The method of claim 12, wherein the inhibitor of PLCβ is U73122, phenylmethylsulfonyl fluoride, manoalide, D609, ET-18-OCH3, compound 48/80 trihydrochloride, spermine tetrahydrochloride, neomycin sulfate, NCDC, or thielavin B.

14. The method of claim 1, wherein the compound is an analogue of PIP2; and the PIP2 analogue is diC8-PIP2.

15. The method of claim 1, wherein the compound increases endogenous PIP2; and the compound inhibits PIP2 hydrolysis or inhibits PIP2 dephosphorylation.

16. The method of claim 1, wherein the cardiovascular disease is a hypertension, arrhythmia, coronary heart disease, cerebrovascular disease, rheumatic heart disease, ventricular hypertrophy, heart failure, vasculitis, atherosclerosis, myocardial infarction, angina pectoris, renal failure, transient ischemic attacks, peripheral vascular disease, aneurysm formation, hypercholesterolemia, hyperlipidemia, hyperlipoproteinemia, deep vein thrombosis, ventricular arrythmia, supraventricular tachycardia, or platelet aggregation.

17. The method of claim 16, wherein the hypertension is pulmonary artery hypertension, systemic hypertension, pulmonary hypertension, sporadic pulmonary arterial hypertension, familial pulmonary arterial hypertension, idiopathic pulmonary arterial hypertension, or acquired pulmonary arterial hypertension.

18. The method of claim 1, wherein the patient has a disease that reduces blood oxygen levels.

19. The method of claim 18, wherein the patient has an infection, inflammation, sepsis, cystic fibrosis, COPD, sleep apnea, or cancer.


SEQUENCE LISTING (9 sequences)

SEQ ID NO: 1 - protein, 4 aa, synthetic construct
  SITE 1: note = (4-hydroxy-3-nitrophenyl)acetyl glutamine
  SITE 4: note = 9-anthracenecarboxylate glycine
QTGG

SEQ ID NO: 2 - protein, 5 aa, synthetic construct
  SITE 1: note = (4-hydroxy-3-nitrophenyl)acetyl glutamine
  SITE 5: note = 9-anthracenecarboxylate glycine
QQTGG

SEQ ID NO: 3 - protein, 4 aa, synthetic construct
  SITE 1: note = (4-hydroxy-3-nitrophenyl)acetyl leucine
  SITE 4: note = 9-anthracenecarboxylate glycine
LRGG

SEQ ID NO: 4 - protein, 4 aa, Homo sapiens
VKKD

SEQ ID NO: 5 - protein, 4 aa, Homo sapiens
VKKN

SEQ ID NO: 6 - protein, 20 aa, Homo sapiens
VHTRQQCRSRFVKKDGHCNV

SEQ ID NO: 7 - protein, 20 aa, Homo sapiens
VHTRRRCRNRFVKKNGQCNI

SEQ ID NO: 8 - protein, 20 aa, Homo sapiens
HVPRRKRRNRFVKKNGQCNV

SEQ ID NO: 9 - protein, 20 aa, Homo sapiens
QSPVGRRRGRFVKKDGHCNV